(19) United States
(12) Patent Application Publication
(10) Pub. No.: US 2023/0309933 A1
(43) Pub. Date: Oct. 5, 2023

(54) SYSTEM AND METHOD FOR ANALYZING USE OF A WEARABLE DEVICE

(71) Applicant: ORPYX MEDICAL TECHNOLOGIES INC., CALGARY (CA)

(72) Inventors: JULIA BREANNE EVERETT, Calgary (CA); RYAN CHARLES LENZ, Calgary (CA); AMANDA ROSE MAXSON, Calgary (CA); LISA MARIE COPPOLA, LITTLETON, CO (US); KRISTINA KAIME HORAK STOHR, SAN LUIS OBISPO, CA (US)

(73) Assignee: ORPYX MEDICAL TECHNOLOGIES INC., CALGARY (CA)

(21) Appl. No.: 18/188,525

(22) Filed: Mar. 23, 2023

Related U.S. Application Data
(60) Provisional application No. 63/326,139, filed on Mar. 31, 2022.

(51) Int. Cl. A61B 5/00; G16H 50/30

(52) U.S. Cl. CPC A61B 5/7271; G16H 50/30; G16H 40/60

(57) ABSTRACT

A system includes a wearable device and a computing device. The wearable device includes one or more sensors for generating first use data corresponding to use of the wearable device by a user. The computing device includes a processor, a memory, and a display device. The processor is configured to receive the first use data; compute a first use metric associated with the user based on the first use data; and compare the first use metric to a first threshold metric. Based on the comparing, the processor is further configured to update a user score by a first score amount; compute a second score amount and a second threshold metric; and provide a first indication of the user score, the second threshold metric, and the second score amount on the display device.

### CROSS-REFERENCES TO RELATED APPLICATIONS

This application claims the benefit of U.S. Provisional Patent Application No. 63/326,139 filed on Mar. 31, 2022, which is incorporated herein by reference in its entirety.

### FIELD

This document relates to systems and methods for analyzing use of a wearable device by a user. In particular, this document relates to receiving use data generated by one or more sensors of the wearable device and computing use metrics associated with the user based on the received use data.

### BACKGROUND

U.S. Patent Publication No. U.S. 2013/0204410 discloses a system and method of tracking physical activity of a person in order to help motivate that person to add more exercise to his/her life. A participant is provided a motion sensor that detects forces incurred by the participant. The motion sensor creates electronic data that corresponds to the forces detected. The data is analyzed to determine whether or not exercise has been performed. The analysis can also determine the type of exercise performed, when the exercise was performed, and the duration of the exercise performed.

U.S. Pat. No. 10,019,552 discloses a method that includes receiving patient information, analyzing the patient information to identify a condition for the patient, formatting a report based on the patient information and the patient condition, and storing at least one of the patient information, the patient condition, and the formatted report as part of a medical record for the patient. The stored information can be processed and analyzed to perform a risk assessment for the patient, as well as compared to other data. Examples of the method may purportedly be used to monitor a medical device from which a communications signal can be sent and received. This may enable patients to enjoy an active lifestyle by not being tied to medical device monitoring equipment that is difficult or impossible to transport or having to routinely visit healthcare facilities. The method may purportedly be used to monitor, process, and transport data from a medical device to a suitable user, such as a healthcare provider.

### SUMMARY

The following summary is intended to introduce the reader to various aspects of the detailed description, but not to define or delimit any invention.

According to some aspects, a computer-implemented method includes: receiving, by a processor, first use data associated with use of at least one wearable device by a user, wherein the first use data includes data generated from one or more sensors of the at least one wearable device; computing, by the processor, a first use metric associated with the user based on the first use data; comparing the first use metric to a first threshold metric; based on the comparing, updating a user score by a first score amount; computing a second score amount and a second threshold metric; and providing, by the processor, a first indication of the user score, the second threshold metric, and the second score amount on a display device.

The method can further include receiving, by the processor, second use data generated by use of the at least one wearable device following receipt of the first indication; computing, by the processor, a second use metric associated with the user based on the second data; comparing the second use metric to the second threshold metric; and when the second use metric satisfies the second threshold metric, updating the user score by the second score amount.

The method may include modifying, by the processor, the second threshold metric in response to computing the second use metric.

The method may include modifying, by the processor, the second threshold metric based on multiple use metrics associated with multiple users.

The method may further include providing to the user, by the processor, a second indication on the display device, wherein the second indication is based on the second use metric and the second threshold metric as modified.

The method may include computing a risk of disengagement for the user, and wherein the computing the second threshold metric is based on the risk of disengagement.

The method may further include receiving, by the processor, a healthcare assessment for the user for a time period associated with use of the at least one wearable device; and modifying, by the processor, the second threshold metric in response to the input and based on at least one of the first use metric and the second use metric.

According to some aspects, a wearable monitoring system includes: at least one wearable device including one or more sensors; and a computing device including a processor, a memory, and a display device. The processor is configured to: receive first use data, the first use data associated with use of at least one wearable device by a user, wherein the first use data comprises data generated from the one or more sensors of the at least one wearable device; compute a first use metric associated with the user based on the first use data; compare the first use metric to a first threshold metric; based on the comparing, update a user score by a first score amount; compute a second score amount and a second threshold metric; and provide a first indication of the user score, the second threshold metric, and the second score amount on the display device.

The processor may be further configured to receive second use data generated by use of the at least one wearable device following receipt of the first indication; compute a second use metric associated with the user based on the second data; compare the second use metric to the second threshold metric; and when the second use metric satisfies the second threshold metric, update the user score by the second score amount.

The processor may be further configured to modify the second threshold metric in response to computing the second use metric.

The processor may be further configured to modify the second threshold metric based on multiple use metrics associated with multiple users.

Modifying the second threshold metric by the processor may include using a machine learning model.

The processor may further be configured to provide to the user a second indication on the display device, wherein the second indication is based on the second use metric and the second threshold metric as modified.

The processor may be further configured to compute a risk of disengagement for the user, and wherein the computing the second threshold metric is based on the risk of disengagement.

The processor may be further configured to receive a healthcare assessment for the user for a time period associated with use of the at least one wearable device; and modify the second threshold metric in response to the input and based on at least one of the first use metric and the second use metric.

In at least some methods and systems, the at least one wearable device includes a sensorized insole.

In at least some methods and systems, the first threshold metric and the second threshold metric may be calibrated to reduce risk factors associated with development of a diabetic foot ulcer.

In at least some methods and systems, the second threshold metric may include a health metric. The health metric may include a maximum activity level.

In at least some methods and systems, the second threshold metric may include a user-specific criterion. The user-specific criterion may include a user-specific activity level.

In at least some methods and systems, the first indication may include at least one of the first use metric, and the second use metric.

In at least some methods and systems, the first use data may indicate one or more of a duration of wear of the at least one wearable device by the user, a number of consecutive or non-consecutive days of use of the at least one wearable device by the user, a duration of wear of the at least one wearable device by the user within a predetermined time interval, a response of the user to one or more alerts provided by the at least one wearable device, a response of the user to one or more indications provided by the processor, an activity level of the user, an interaction level of the user with a digital platform associated with the at least one wearable device, a use level of a wireless charger for the at least one wearable device by the user, and a care level of the at least one wearable device by the user.

In at least some methods and systems, the one or more sensors of the at least one wearable device may include at least one of a force sensor and a temperature sensor.

According to some aspects, the present disclosure provides a non-transitory computer-readable medium storing computer-executable instructions. The computer-executable instructions, when executed, configure a processor to perform any of the methods described herein.

According to some aspects, a computer-implemented method includes: receiving sensor data and use data associated with use of at least one wearable device by a user, wherein the sensor data and the use data comprise data generated from one or more sensors of the at least one wearable device; generating, by a processor, monitoring data generated from monitoring the sensor data and the use data of the at least one wearable device of the user; comparing a numeric value of a day of a specified period to a numeric value of a predefined day of the specified period; computing, by the processor, a use metric based on the sensor data or the use data or a monitoring metric based on the monitoring data, if the numeric value of the day of the specified period is greater than or equal to the numeric value of the predefined day of the specified period; comparing the use metric or the monitoring metric to a threshold metric; determining whether the use metric or the monitoring metric satisfies a threshold requirement of the threshold metric; and generating an alert if the use metric or the monitoring metric does not satisfy the threshold requirement.

The method can further include clearing the alert.

The alert can be cleared only if the numeric value of the day of the specified period is less than the numeric value of the predefined day of the specified period, if the use metric or the monitoring metric satisfies the threshold requirement, or if there is a request to clear the alert.

The monitoring metric can be a monthly remote patient monitoring time total or a monthly number of practitioner-user interactions.

The use metric can be a number of consecutive or non-consecutive days of use.

The numeric value of the predefined day can be 20, and the specified period can be a month.

The method can further include updating the threshold metric based on the use metric or the monitoring metric.

The method can further include computing a risk of disengagement for the user based on the use metric or the monitoring metric.

According to some aspects, a wearable monitoring system includes: at least one wearable device including one or more sensors; and a computing device including a processor, a memory, and a display device. The processor is configured to: receive sensor data and use data associated with use of the at least one wearable device by a user, wherein the sensor data and the use data comprise data generated from the one or more sensors of the at least one wearable device; generate monitoring data, the monitoring data generated from monitoring the sensor data and the use data of the at least one wearable device of the user; compare a numeric value of a day of a specified period to a numeric value of a predefined day of the specified period; compute a use metric based on the sensor data or the use data or a monitoring metric based on the monitoring data, if the numeric value of the day of the specified period is greater than or equal to the numeric value of the predefined day of the specified period; compare the use metric or the monitoring metric to a threshold metric; determine whether the use metric or the monitoring metric satisfies a threshold requirement of the threshold metric; and generate an alert on the display device if the use metric or the monitoring metric does not satisfy the threshold requirement.

The processor can be further configured to clear the alert.

The alert can be cleared only if the numeric value of the day of the specified period is less than the numeric value of the predefined day of the specified period, if the use metric or the monitoring metric satisfies the threshold requirement, or if there is a request to clear the alert.

The monitoring metric can be a monthly remote patient monitoring time total or a monthly number of practitioner-user interactions.

The use metric can be a number of consecutive or non-consecutive days of use.

The numeric value of the predefined day can be 20, and the specified period can be a month.

The processor can be further configured to update the threshold metric based on the use metric or the monitoring metric.

The processor can be further configured to compute a risk of disengagement for the user based on the use metric or the monitoring metric.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is a block diagram illustrating an example of a wearable monitoring system for analyzing data generated by use of a wearable device by a user;

FIG. 2 is a diagram illustrating an example of a wearable device incorporating a sensing unit that can be used in the wearable monitoring system of FIG. 1;

FIG. 3 is a block diagram illustrating an example of a processing device that can be used in the wearable monitoring system of FIG. 1;

FIG. 4 is a flowchart illustrating an example of a method for analyzing data generated by use of a wearable device by a user;

FIG. 5 is a flowchart illustrating another example of a method for analyzing data generated by use of a wearable device by a user;

FIG. 6 is a flowchart illustrating another example of a method for analyzing data generated by use of a wearable device by a user; and

FIG. 7 is a flowchart illustrating another example of a method for analyzing data generated by use of a wearable device by a user.

### DETAILED DESCRIPTION

Various apparatuses or processes or compositions will be described below to provide an example of an embodiment of the claimed subject matter. No embodiment described below limits any claim and any claim may cover processes or apparatuses or compositions that differ from those described below. The claims are not limited to apparatuses or processes or compositions having all of the features of any one apparatus or process or composition described below or to features common to multiple or all of the apparatuses or processes or compositions described below. It is possible that an apparatus or process or composition described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

For simplicity and clarity of illustration, reference numerals may be repeated among the figures to indicate corresponding or analogous elements. In addition, numerous specific details are set forth in order to provide a thorough understanding of the subject matter described herein. However, it will be understood by those of ordinary skill in the art that the subject matter described herein may be practiced without these specific details. In other instances, well-known methods, procedures and components have not been described in detail so as not to obscure the subject matter described herein. The description is not to be considered as limiting the scope of the subject matter described herein.

The terms “coupled” or “coupling” as used herein can have several different meanings depending in the context in which these terms are used. For example, the terms coupled or coupling can have a mechanical, electrical or communicative connotation. For example, as used herein, the terms coupled or coupling can indicate that two elements or devices are directly connected to one another or connected to one another through one or more intermediate elements or devices via an electrical element, electrical signal, or a mechanical element depending on the particular context. Furthermore, the term “communicative coupling” may be used to indicate that an element or device can electrically, optically, or wirelessly send data to another element or device as well as receive data from another element or device.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Terms of degree such as “substantially”, “about”, and “approximately” as used herein mean a reasonable amount of deviation of the modified term such that the end result is not significantly changed. These terms of degree may also be construed as including a deviation of the modified term if this deviation would not negate the meaning of the term it modifies.

Any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

Some elements herein may be identified by a part number, which is composed of a base number followed by an alphabetical or subscript-numerical suffix (e.g. 112a, or 1121). Multiple elements herein may be identified by part numbers that share a base number in common and that differ by their suffixes (e.g. 1121, 1122, and 1123). All elements with a common base number may be referred to collectively or generically using the base number without a suffix (e.g. 112).

The systems, methods, and devices described herein may be implemented as a combination of hardware or software. In some cases, the systems, methods, and devices described herein may be implemented, at least in part, by using one or more computer programs, executing on one or more programmable devices including at least one processing element, and a data storage element (including volatile and non-volatile memory and/or storage elements). These devices may also have at least one input device (e.g. a pushbutton keyboard, mouse, a touchscreen, and the like), and at least one output device (e.g. a display screen, a printer, a wireless radio, and the like) depending on the nature of the device.

Some elements that are used to implement at least part of the systems, methods, and devices described herein may be implemented via software that is written in a high-level procedural language such as object-oriented programming. Accordingly, the program code may be written in any suitable programming language such as Python or C, for example. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods described herein may be capable of being distributed in a computer program product including a computer readable medium that bears computer usable instructions for one or more processors. The medium may be provided in various forms, including non-transitory forms such as, but not limited to, one or more diskettes, compact disks, tapes, chips, and magnetic and electronic storage. Alternatively, the medium may be transitory in nature such as, but not limited to, wire-line transmissions, satellite transmissions, internet transmissions (e.g. downloads), media, digital and analog signals, and the like. The computer useable instructions may also be in various formats, including compiled and non-compiled code.

Described herein are systems, methods and devices for analyzing use of at least one wearable device by a user. The wearable device can be a device designed to improve health, decrease risk factors associated with certain health conditions, and/or improve fitness outcomes of the user. A health outcome can be a change in health status of a user or a health-related event that is a result of healthcare interventions. In some examples, the wearable device may include a medical device used to help with acute or chronic disease management. Proper and consistent use of the wearable device may serve to improve the efficiency of the wearable device in improving health and/or fitness outcomes.

As an example, the wearable device may be a wearable insole device used to help manage user conditions associated with diabetes. Peripheral neuropathy is the damage of peripheral nerves, which may cause weakness, decreased sensation, pain, numbness and ulcers in extremities for users suffering from diabetes. In patients with peripheral neuropathy, diabetic foot ulcers (DFUs) represent a significant source of patient morbidity, healthcare cost, and are associated with increased mortality. Wearable insole devices may be used to prevent or reduce the likelihood and severity of DFUs. Proper and consistent use of the wearable insole device may serve to improve the efficiency of the wearable insole device in reducing DFU occurrence.

In the systems, methods, and devices disclosed herein, the wearable device includes one or more sensors. The sensors may be configured to collect health and/or fitness data used to improve the health and/or fitness outcome of a user. For example, a wearable insole device may include pressure (or force) and temperature sensors that can detect high pressure or high temperature zones on a user's foot. The collected pressure and/or temperature sensor data can be used to identify and indicate early warning signs of DFUs.

The data collected by the sensors may also be used to compute use metrics indicating use of the wearable device by the user. For example, the described systems, methods and devices can use the data collected by the pressure and temperature sensors of a wearable insole device to compute use metrics like duration of use by the user.

In some examples, the data collected by the sensors may be combined with other use data that can provide further insight into use of the wearable device. For example, there may be a digital platform or app associated with the wearable device. The other use data can include interaction data of the user with the digital platform or app. The other use data can also include data indicating, for example, responsiveness of the user to one or more alerts provided by the wearable device, usage of a wireless charger for the wearable device, and the user's care level of the wearable device.

The computed use metrics can indicate whether the wearable device is being used properly and/or consistently. For the example wearable insole device, the computed use metrics may include duration of wear of the wearable device by the user, a number of consecutive or non-consecutive days of use of the wearable device by the user, a duration of wear of the wearable device by the user within a predetermined time interval, a response of the user to one or more alerts provided by the wearable device, a response of the user to one or more indications provided by the processor, an activity level of the user, an interaction level of the user with a digital platform associated with the wearable device, a use level of a wireless charger for the wearable device by the user, care personnel inputs, and a care level of the wearable device by the user.

Use metrics can be computed based on collected sensor data. The collected sensor data is verifiable data that can accurately reflect the use of the wearable device by the user. Conversely, non-verifiable data, like data self-reported by the user, may not accurately reflect the use of the wearable device by the user. For example, the non-verifiable data may include data entered by a user into a digital app on a smartphone device. A user may forget to enter a portion of the data or in some examples, enter inaccurate data (e.g., a user may overestimate the time they have used the wearable device). Self-reported data may also cause user disengagement or user fatigue when users become tired of repeatedly entering use data over time. The described systems, methods and devices can in some examples avoid or mitigate these disadvantages by using verifiable data collected by the sensors of the wearable device. In some cases, non-verifiable data may also be used to enhance user interaction with the wearable device. Similarly, positive feedback (e.g., notification messages, splash animations, etc.) may enhance user interaction with the wearable device.

Monitoring data may be generated by a monitoring device, such as a processor in communication with the wearable device and can be used to generate monitoring metrics. Monitoring metrics are metrics that represent a level or quality of monitoring. Monitoring metrics may be generated or reviewed by an additional user or a monitoring device that monitors the sensor data, use data, and/or use metrics of the user of the wearable device. The additional user may be a healthcare practitioner, a program administrator, or a program provider. For example, a healthcare practitioner may provide remote patient monitoring to a wearable device user. With remote patient monitoring, data collected from a user's wearable device can be sent to a healthcare practitioner (e.g. a nurse, a doctor, etc.) or a monitoring device for real-time or post-hoc analysis. The healthcare practitioner can follow up with the user if a concern is identified in the data or an alert can be generated by the monitoring device. Remote patient monitoring allows healthcare practitioners to efficiently assess users in their real-life environments and identify health risks as they occur, allowing for timely interventions. Monitoring metrics for a healthcare practitioner providing remote patient monitoring to a user may include remote patient monitoring time (i.e. time spent reviewing sensor data, use data, or use metrics) and monthly number of practitioner-user interactions. These example monitoring metrics provide a quantitative indication of the quality of remote patient monitoring that may be provided by the healthcare practitioner to a user. The monitoring metrics may trigger the provision of alerts to a monitoring device which monitors multiple wearable devices.

The described systems, methods and devices can compare the computed use metrics and/or monitoring metrics to one or more threshold metrics. The threshold metrics can be evidence-based targets for use of the wearable device to improve health and/or fitness outcomes. For example, if medical studies indicate that six hours of daily use of the example wearable insole device can improve a user's DFU-related health outcome, one of the threshold metrics associated with the use metric of daily hours of use of the wearable insole device can be an improved outcome threshold metric of six hours. At least one medical study has found that at least 4.5 hours of daily activity may improve DFU-related health outcomes. The threshold metrics can be set by a healthcare practitioner, program administrator, or program provider. For example, the threshold metrics can be personalized targets prescribed by a healthcare practitioner. In another example, the threshold metrics can be set by a program administrator to encourage users to meet medical billing requirements. The threshold metrics can also be personalized targets generated by the monitoring device.

The described systems, methods and devices can enable multiple threshold metrics associated with each use metric or monitoring metric, e.g., a baseline threshold metric, an improved outcome threshold metric and a maximum threshold metric. For the example use metric of daily hours of use of the wearable insole device, the baseline threshold metric may be 3 hours, the improved outcome threshold metric may be 4.5 hours, and the maximum threshold metric may be 6 hours. In some cases, the threshold metrics may be determined based on other metrics, such as number of steps. For example, in a patient with a recent DFU occurrence, a maximum threshold metric may be, e.g., 3000 steps to avoid a further breakdown of recently healed tissue.

Multiple threshold metrics may encourage incremental improvements in usage of the wearable device compared with a single threshold metric. A single threshold metric that is too high may cause disengagement of users unable to achieve the threshold metric within a short time period. In some cases, a very low threshold metric may also increase disengagement by reducing a user's enjoyment or sense of accomplishment at achieving the threshold metric. Incremental threshold metrics may also help a new user establish proper and consistent usage habits that can improve the effectiveness of the wearable device in improving the health and/or fitness outcomes. In some examples, the multiple threshold metrics may be incremented on a linear scale. In some examples, the multiple threshold metrics may be incremented on an exponential scale.

For an example use metric, the maximum threshold metric can be an evidence-based target to prevent overuse of the wearable device, which may cause injuries or other problems. For example, an improved outcome threshold metric may correspond to a use metric level for which the wearable device provides a high level of efficiency in improving health and/or fitness outcomes. Further use of the wearable device, between the improved outcome threshold metric and the maximum threshold metric, may correspond to diminishing levels of efficiency of the wearable device in improving health and/or fitness outcomes. Beyond the maximum threshold metric, a risk of injury may outweigh any incremental benefits in improving health and/or fitness outcomes.

The described systems, methods and devices can compare the computed use metrics to corresponding threshold metrics and update a user score if the computed use metric exceeds a threshold metric. For the example use metric of daily hours of use of the wearable insole device and an associated threshold metric of 4.5 hours, the user score can be updated if the user uses the wearable insole for more than 4.5 hours in a day. In another example, wherein the use metric is non-consecutive days of use of the wearable insole device and the associated threshold metric is 16 non-consecutive days of use, the user score can be updated if the user uses the wearable insole for more than 0.9 hours in a day for 16 non-consecutive days of use in a month.

The updated user score can be used to provide positive incentives to the user encouraging the user to maintain proper and consistent use of the wearable device, and/or further improve the user's usage of the wearable device. For example, the user score may be used to provide monetary incentives that are in compliance with applicable laws and regulations. In some examples, other incentives like gifts, discounted goods or services, reduced insurance premiums, monetary donations (e.g., to charitable causes), loyalty program points, or digital game assets like skins, characters, levels etc. may be provided. This can incentivize improved or consistent usage of the wearable device and result in improved efficiency of the wearable device in improving the user's health and/or fitness outcomes.

The positive incentives may be provided by a program provider or a program administrator. A program provider may be a corporation, an organization or an individual associated with the wearable device and with an objective to improve the efficiency of the wearable device in improving health and/or fitness outcomes. A program administrator may be a corporation, an organization or an individual associated with the wearable device and responsible for the operation of one or more systems or methods described herein. In some cases, the program provider and program administrator may be the same corporation, organization, or individual. The positive incentives may also be provided by the system.

The described systems, methods and devices can provide real-time notifications to the user regarding updated user scores and associated threshold metrics. The real-time notifications may incentivize further proper use of the wearable device by the user compared with a system where there is a long delay between the data input and the notification (e.g., a user self-reporting data from previous days that is then reviewed before the user score is updated and the user is notified).

In some examples, the described systems, methods and devices can update the threshold metrics based on one or more use metrics computed for a user. For example, a threshold metric may be reduced to encourage usage for a user who appears to be at risk of being disengaged from using the wearable device. This may help reduce disengagement occurrence among users, improve usage of the wearable device and therefore improve the efficiency of the wearable device in improving the user's health and/or fitness outcome.

The described systems, methods and devices can enable customized threshold metrics for each user. For example, a user's disengagement risk score can be computed during initial registration and customized threshold metrics can be determined to reduce disengagement risk. This provides an advantage over using non-customizable threshold metrics for the entire population of users that may work well for a portion of the users but may result in disengagement of many other users.

The user's disengagement risk score can be computed based on inputs from a healthcare practitioner, program administrator, or program provider. The inputs may include various risk categories, which are assessed by the healthcare practitioner, program administrator or program provider. The inputs may indicate the highest overall health and/or fitness concerns for the individual user and the threshold metrics may be customized based on those concerns. For example, mental health and obesity may be selected as concerns for a user. The threshold metrics may then be higher for the step count use metric, to encourage physical activity.

In some examples, a user's disengagement risk score can be computed based on the trend of the user's use metrics. Threshold metrics that may have been computed during initial registration of user can be changed if a recent trend of the user's use metrics indicates a high risk of disengagement. This can provide an advantage over using static threshold metrics that may work initially but result in disengagement over a period of time.

In some examples, a user's disengagement risk score can be computed based on the trend of the user's monitoring metrics. For example, interactions between a healthcare practitioner and a user can remind and positively reinforce the user to keep engaging with the wearable device, lessening their risk of disengagement. However, too frequent interactions can be disruptive to the user and can increase their risk of disengagement. Therefore, if a recent trend of the user's use metrics indicate a high risk of disengagement, their level or quality of monitoring can be adjusted to decrease this risk.

The described systems, methods and devices can also be used to analyze inputs from multiple wearable devices. Examples of other wearable devices that may be used include, for example, a heart rate monitor wearable device and/or a blood glucose monitoring device. The inputs from these additional devices can be combined with inputs from, e.g., an insole device for a user to be analyzed using a single system, method or device. For example, a unified system may include a wearable insole to monitor DFU development and physical activity, and a blood content monitor (e.g. for monitoring glucose, electrolytes, minerals, oxygen, carbon dioxide, carbon monoxide, HbA1C, Ethanol, protein, lipid, carbohydrate, cortisol, lactate, pH, pro- and anti-inflammatory markers, MMPs, Growth Factors, bacterial content) to measure blood content levels. The user may be rewarded based on various thresholds for each device. The user's user score may be increased based on daily use time, consecutive and non-consecutive days of use, wireless charging use, and various other use metrics and threshold metrics using sensor data collected from the wearable insole. The user may also increase their user score based on blood glucose levels, frequency of use, and various other use metrics and threshold metrics using sensor data collected from the blood glucose monitor. The user scores from each device may be cumulated.

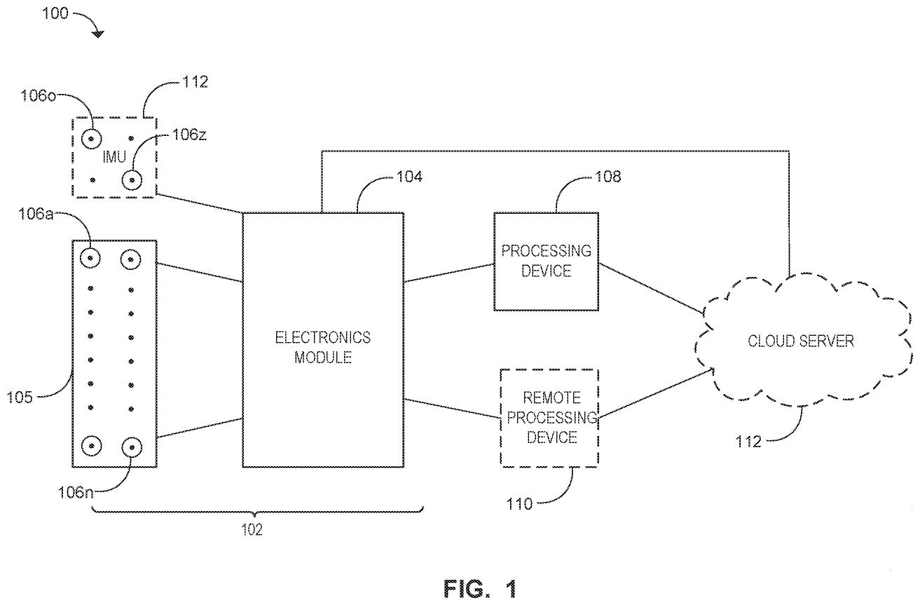

Referring now to FIG. 1, shown therein is a block diagram illustrating an example wearable monitoring system 100 that can be used to analyze use data generated by use of a wearable device by a user. System 100 includes an input unit 102 (also referred to herein as an input device), a processing device 108 (also referred to herein as a receiving device or an output device), an optional remote processing device 110 and an optional remote cloud server 112.

Input unit 102 generally includes a sensing unit 105 and an electronics module 104. Sensing unit 105 can include a plurality of sensors 106a-106n. Optionally, input unit 102 can include an inertial measurement unit (IMU) 112. As will be described in further detail below, the input unit 102 may for example be combined with, or integrated into, the wearable device.

The wearable device can be manufactured of various materials such as fabric, cloth, polymer, or foam materials suitable for being worn close to, or in contact with, a user's skin. All or a portion of the wearable device may be made of breathable materials to increase comfort while a user is performing an activity. In some examples, however, the wearable device may be an implantable device that is inserted within the user's body. In still other examples, the wearable device may be designed for intermittent use, similar to non-continuous blood glucose monitoring devices.

In some examples, the wearable device may be formed into a garment or form of apparel such as a sock, a shoe, or an insole. Some wearable devices such as socks may be in direct contact with a user's skin. Some wearable devices, such as shoes, may not be in direct contact with a user's skin but still positioned within sufficient proximity to a user's body to allow sensors integrated into the wearable device to acquire the health, fitness or use data.

In some examples, the wearable device may be a compression-fit garment. The compression-fit garment may be manufactured from a material that is compressive. A compression-fit garment may minimize the impact from “motion artifacts” by reducing the relative movement of the wearable device with respect to a target location on the individual's body. In some examples, the wearable device may also include anti-slip components on the skin-facing surface. For example, a silicone grip may be provided on the skin-facing surface of the wearable device to further reduce the potential for motion artifacts.

The wearable device can be worn on a foot. For example, the wearable device may be a shoe, a sock, or an insole, or a portion of a shoe, a sock, or an insole. The wearable device may include a deformable material, such as foam. This may be particularly useful where the wearable device is a shoe or insole.

Sensors 106a-106n of sensing unit 105 can be configured to collect a user's health and/or fitness data. For example, sensors 106a-106n can collect data including measurements of a user's heart rate, pulse rate, beat-to-beat heart variability, EKG or ECG, respiration rate, skin temperature, core body temperature, heat flow off the body, galvanic skin response (GSR), EMG, EEG, EOG, blood pressure, body fat, hydration level, activity level, oxygen consumption, glucose or blood sugar level, body position, pressure on muscles or bones, and/or UV radiation absorption.

IMU 112 can include one or more sensors 106o-106z for measuring the position and/or motion of a user. For example, IMU 112 may include sensors such as one or more of a gyroscope, accelerometer (e.g., a three-axis accelerometer), magnetometer, orientation sensor (for measuring orientation and/or changes in orientation), angular velocity sensor, and inclination sensor. Generally, IMU 112 includes at least an accelerometer. One or more of sensors 106a-106z may also be referred to herein as sensors 106.

The wearable device can be configured to position sensors 106 in contact with (or in close proximity to) a user's body to allow sensors 106 to measure an aspect of the activity being performed by the user. Sensors 106 may be configured to measure a particular sensed variable at a location of a user's body when the wearable device is engaged with the user's body. Sensors 106 can be integrated into the material of the wearable device. Alternatively, sensors 106 can be affixed or attached to the wearable device, for example, printed, glued, laminated or ironed onto a surface, or between layers, of a wearable device.

In some examples, sensors 106a-106n of sensing unit 105 and sensors 106o-106z of IMU 112 may be provided by the same wearable device. Alternatively, sensors 106a-106n and sensors 106o-106z may be provided by different wearable devices.

For clarity and ease of explanation, the below description refers to a wearable device in the form of an insole device. The insole device may be provided in various forms, such as an insert for footwear, or integrated into a shoe. In many cases, users may be provided with two insole devices corresponding to the user's feet. However, the systems and methods described herein can be implemented using other wearable devices, for example, the other wearable devices described herein.

In particular, although not illustrated in FIG. 1, wearable devices may include, e.g., heart rate monitors, blood glucose monitors, and other monitoring devices. Each wearable device may have its own input unit 102, which is operatively connected to processing device 108 and/or remote processing device 110.

Sensors 106 of the wearable insole device (also referred to as “sensels” or sensing elements) may include one or more force sensors. Various types of force sensors may be used, such as force sensing resistors, pressure sensors, piezoelectric tactile sensors, elasto-resistive sensors, capacitive sensors or more generally any type of force sensor that can be integrated into a wearable device.

Sensors 106a-106n can be configured to collect force sensor data from underneath an individual's foot. IMU 112 can also be positioned underneath an individual's foot. However, the IMU 112 need not be positioned underfoot so long as the IMU 112 can collect inertial measurement data relating to the position and/or motion of the foot.

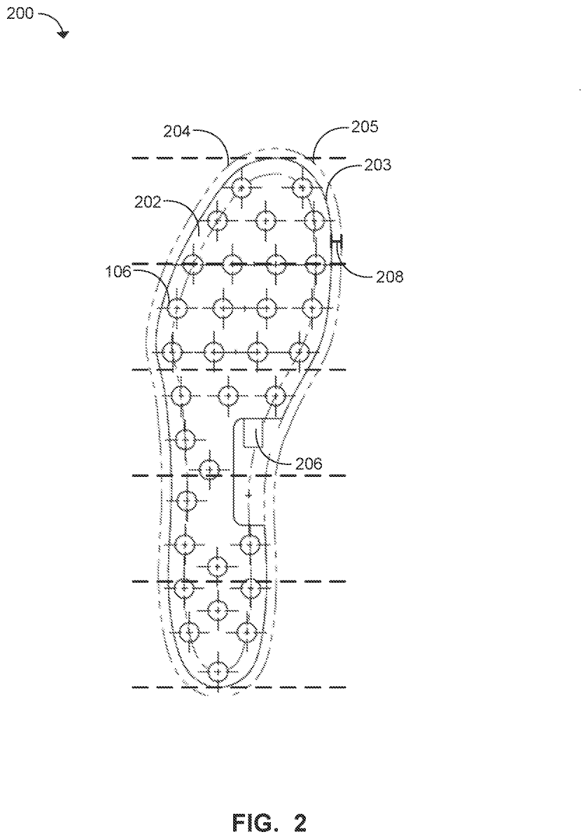

Sensors 106 may be arranged into a sensor array. As used herein, the term sensor array refers to a series of sensors arranged in a defined grid. Sensors 106 can be arranged in various types of sensor arrays. For example, sensors 106 can be provided as a set of discrete sensors (see e.g., FIG. 2). A discrete sensor is an individual sensor that acquires a sensor reading at a single location. A set of discrete sensors generally refers to multiple discrete sensors that are arranged in a spaced apart relationship in a sensing unit.

Sensors 106 may be arranged in a sparse array of discrete sensors that includes void locations where no sensors 106 are located. Alternatively, sensors 106 may be arranged in a continuous or dense sensor array in which sensors 106 are arranged in a continuous, or substantially continuous manner, across the grid.

Discrete sensors can provide an inexpensive alternative to dense sensor arrays for many applications. However, because no sensors are positioned in the interstitial locations between the discrete sensors and the void locations external to the set of discrete sensors, no actual sensors readings can be acquired for these locations. Accordingly, depending on the desired resolution for the force sensor data, sensor readings may be estimated (rather than measured) at the interstitial locations and at the void locations external to the set of discrete sensors in order to provide sensor data with similar resolution to a dense sensor array. Alternatively, where lower resolution force sensor data is sufficient, sensor readings may not necessarily be estimated.

As shown in FIG. 1, input unit 102 includes electronics module 104 coupled to the sensors 106 and to optional IMU 112. In some examples, electronics module 104 can include a power supply, a controller or other processing unit, a memory, a signal acquisition unit operatively coupled to the controller and to the sensors 106 (and to IMU 112), and a wireless communication module operatively coupled to the controller.

Generally, the sensing unit refers to the sensors 106 and the signal acquisition unit. The signal acquisition unit may provide initial analog processing of signals acquired using sensors 106, such as amplification. The signal acquisition unit may also include an analog-to-digital converter to convert the acquired signals from the continuous time domain to a discrete time domain. The analog-to-digital converter may then provide the digitized data to the controller for further analysis or for communication to processing device 108, remote processing device 110 and/or remote cloud server 112 for further analysis.

Optionally, electronics module 104 may include a controller or other processing device configured to perform the signal processing and analysis. In such cases, the controller on electronics module 104 may be configured to process the received sensor readings in order to analyze the sensor data. In some examples, the controller may be coupled to the communication module (and thereby the sensing unit) using a wired connection such as Universal Serial Bus (USB) or other port.

Electronics module 104 can be communicatively coupled to processing device 108, remote processing device 110 and/or remote cloud server 112. The communication may include using a wireless communication module (e.g., Bluetooth, Bluetooth Low-Energy, Wi-Fi, ANT+IEEE 802.11, etc.) and/or a wide area network like the Internet.

Processing device 108 and/or remote processing device 110 can be any type of processing device such as (but not limited to) a personal computer, a tablet, and a mobile device such as a smartphone, a smartwatch or a wristband. In some examples, input unit 102 and processing device 108 may be integrated into a single unit or device.

Each processing device 108, remote processing device 110 and/or remote cloud server 112 typically includes a processor unit, an output device (such as a display, speaker, and/or tactile feedback device), a user interface, an interface unit for communicating with other devices, Input/Output (I/O) hardware, a wireless unit (e.g. a radio that communicates using CDMA, GSM, GPRS or Bluetooth protocol according to standards such as IEEE 802.11a, 802.11b, 802.11g, or 802.11n), a power unit, and a memory unit. The memory unit can include RAM, ROM, one or more hard drives, one or more flash drives, or some other suitable data storage elements such as disk drives, etc.

The processor unit controls the operation of processing device 108, remote processing device 110 and/or remote cloud server 112 and can be any suitable processor, controller or digital signal processor that can provide sufficient processing power processor depending on the desired configuration, purposes, and requirements of system 100.

The display can be any suitable display that provides visual information. For instance, the display can be a cathode ray tube, or a flat-screen monitor and the like if processing device 108, remote processing device 110 and/or remote cloud server 112 is a desktop computer. In other examples, the display can be a display suitable for a laptop, tablet, or handheld device, such as an LED or LCD-based display and the like. The display may be remote to the wearable device.

System 100 can generally be used to analyze sensor data for various purposes. For the example wearable insole device, system 100 can be used to analyze force sensor data for various purposes such as identifying foot contact events, foot contact periods and ground reaction forces based on sensor readings received from a plurality of sensors positioned underfoot. In some examples, system 100 may also track additional data derived from the sensor readings. The sensor readings, foot contact event data, foot contact period data, ground reaction force data, and derived data may be monitored, stored, and analyzed for the user. Aspects of the monitoring, storage and analysis of biometric features and other metrics may be performed by one or more of input unit 102, processing device 108, remote processing device 110, and/or remote cloud server 112. For example, a non-transitory storage memory of one or more of the input unit 102, processing device 108, remote processing device 110, and/or remote cloud server 112 can store a machine learning model trained to predict ground reaction forces.

Remote cloud server 110 may provide additional processing resources not available on input unit 102, processing device 108, or remote processing device 110. For example, some aspects of processing the sensor readings acquired by sensors 106 may be delegated to remote cloud server 112 to conserve power resources on input unit 102 or remote processing device 110. In some cases, remote cloud server 112, input unit 102, processing device 108 and/or remote processing device 110 may communicate in real-time to provide timely feedback to a user regarding the sensor readings, foot contact event data, foot contact period data, ground reaction force data, and other related data.

In the example system 100 illustrated in FIG. 1, a single input unit 102 is shown. However, system 100 may include multiple input units 102 associated with the same individual. For the example wearable insole devices, system 100 may include two separate input units 102, each input unit 102 associated with one of the individual's legs. Sensor data from an individual input unit 102 may be used for analysis of the force sensor data for the individual's corresponding leg. Accordingly, system 100 may include a separate sensing unit 105 (and a separate IMU 112 where IMU 112 is included in system 100) for each foot of an individual. This may allow the force sensor data to be determined separately for each of the individual's feet.

Referring now to FIG. 2, shown therein is an example of an insole device 200 that includes a sensing unit 202. Insole device 200 is an example of an input unit 102 that may be used in system 100 shown in FIG. 1. Insole device 200 may be the footwear insert described in PCT Application No. PCT/CA2020/051520 (Everett et al.), the entirety of which is incorporated herein by reference.

Insole device 200 includes a sensing unit 202 and an optional liner 204. Liner 204 can provide a protective surface between sensing unit 202 and an individual's foot. Liner 204 may have a slightly larger profile as compared to sensing unit 202. That is, the outer perimeter 203 of sensing unit 202 may be inwardly spaced from the outer perimeter 205 of liner 204 by an offset 208. The offset 208 may be substantially consistent throughout the perimeter of sensing unit 202 such that sensing unit 202 is completely covered by liner 204.

Optionally, insole device 200 can include an IMU (not shown). Insole device 200 can also include a connector 206. Connector 206 may provide a coupling interface between sensors 106 (and the optional IMU) and an electronics module (not shown) such as electronics module 104. The coupling interface can allow signals from sensors 106 and/or IMU to be transmitted to the electronics module. In some examples, the coupling interface may also provide control or sampling signals from the electronics module to sensors 106 and/or the IMU.

The arrangement of sensors 106 in sensing unit 202 is an example of a sparse sensor array that may be used to collect force sensor data. In alternative examples, various different types of force sensors, force sensor arrays, and arrangements of force sensors may be used. For example, sensor units containing a dense force sensor array (e.g., a Pedar® insole with 99 sensors or Tekscan® system) may also be used.

Incorporating sensing unit 202 in a wearable device such as insole device 200 may provide advantages over incorporating the sensing unit in, for example, fitness equipment. For example, including sensing unit 202 in the wearable device (e.g., insole device 200) does not require any special installation or modifications to the fitness equipment used by an individual.

Incorporating the sensing system into a wearable device may also help reduce the cost of the sensing system. Furthermore, the wearable device may allow an individual to measure pressure and other sensor data (e.g., IMU sensor data) while performing various activities including running, walking, jumping, cycling etc.). This can further offset the cost of the sensing system, as a single sensing system may be used for multiple activities, rather than requiring separate specialized sensing systems for each activity.

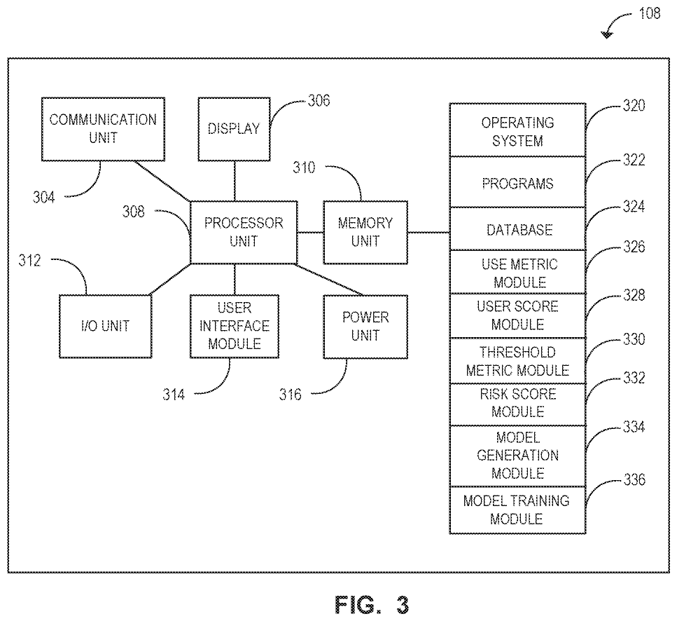

Referring now to FIG. 3, shown therein is a block diagram illustrating an example processing device 108 that can be used in a wearable monitoring system to analyze use data generated by use of a wearable device by a user. In the example illustrated, processing device 108 includes a communication unit 304, a display 306, a processor unit 308, a memory unit 310, an I/O unit 312, a user interface module 314, and a power unit 316.

Communication unit 304 can include wired or wireless connection capabilities. Communication unit 304 can be used by processing device 108 to communicate with other devices or computers. For example, processing device 108 can use communication unit 304 to receive sensor data from electronics module 104 or to provide notifications to a remote processing device 110 operated by the user. In other examples, processing device 108 can use communication unit 304 or to receive historical wearable device use data or use data for other users from cloud server 112.

Processor unit 308 can control the operation of processing device 108. Processor unit 308 can be any suitable processor, controller or digital signal processor that can provide sufficient processing power depending on the configuration, purposes and requirements of processing device 108 as is known by those skilled in the art. For example, processor unit 308 may be a high-performance general processor. For example, processor unit 308 may include a standard processor, such as an Intel® processor, or an AMD® processor. Alternatively, processor unit 308 can include more than one processor with each processor being configured to perform different dedicated tasks. Alternatively, specialized hardware can be used provide some of the functions provided by processor unit 308.

Processor unit 308 can execute a user interface module 314 that is used to generate various user interfaces. User interface module 314 may be configured to provide a user interface on display 306. Optionally, processing device 108 may be in communication with external displays and user interface module 314 may also generate user interfaces for the external displays that are in communication with processing device 108.

User interface module 314 can be configured to provide a user interface for displaying computed use metrics, monitoring metrics, alerts, threshold metrics and/or user scores to a user. The user interface can include data output display portions showing the metrics and scores to a user. The data output display may include graphs or charts showing the numerical values and/or progress towards a threshold metric or a user-defined goal. In some examples, gamification characteristics may be added to the data output display. For example, a daily use metric of a wearable device may be displayed as a tracking ring that closes as the user progresses towards a daily use goal. A visual alert and user score update notification can be provided when the tracking ring completely closes indicating that the user has completed the daily use goal. In some examples, the user interface may provide a daily, weekly, or monthly leaderboard showing data for the user and a related group of other users. In some examples, users can be grouped into teams and the user interface can display use data for the different teams. Alternatively, the user may opt not to use the interface module 314, and instead communicate directly with a healthcare practitioner, program administrator or program provider to discuss program details including use metrics, the threshold metrics and/or user scores, either in person or via telephone.

User interface module 314 may also include audible or haptic displays to alert the user to any computed use metrics, monitoring metrics, threshold metrics and/or user scores. For example, when the user has reached a threshold metric for the daily use metric, a vibration may be emitted to the user. User interface module 314 may also display reports with various statistics and trends in a user's calculated or collected data. The reports may be generated by data analytics and reporting software (e.g. Power BI® software owned by Microsoft Corporation of Washington, USA). The data analytics and reporting software may be used to further analyze the use metrics, monitoring metrics, alerts, threshold metrics and/or user scores to generate the reports. The reports may then be reviewed by the healthcare practitioner, program provider, program administrator, user, or monitoring device. User interface module 314 may also include a medication dose calculator for calculating the medication needs of the user. In the example, the medication dose calculator may depend on sensor data, use data, use metrics, threshold metrics, alerts, monitoring metrics, and/or monitoring data. The medication calculator and medication needs may be displayed or passed to an automated medication delivery system.

The user interface provided by user interface module 314 can also include user input portions operable to receive input from users. For example, a user may input their demographic and other information or provide answers to a questionnaire using the input portion of the user interface. A user may also provide inputs in response to user score update notifications.

User interface module 314 may also be configured to provide different incentive visuals to a user, for example, tiers, badges or medals that may be displayed in association with the user on a digital platform; a congratulatory message, an abstract graphical representation such as a bar chart, a progressive animation (e.g., leaves added to a tree), and/or a graphic representation of a healing foot ulcer that visually heals with ongoing use of the wearable device.

User interface module 314 may also be used to monitor the sensor data, use data, and/or use metrics of a user of a wearable device. For example, the user interface module 314 may be provided as an online dashboard, with which a healthcare practitioner can access the data from the user. In the example, the online dashboard may be used to monitor one or more wearable device users. User interface module 314 may also be connected to a telephone, electronic mailbox, or web-based communication application (e.g. the Webex® video conferencing application owned by Cisco Systems, Inc. of San Jose California, USA) to record data from practitioner-user interactions. For example, user interface module 314 may record the length of time a healthcare practitioner spends on a web-based communication application talking to a user, wherein the user is identified by a telephone number linked to the user.

The user interface provided by user interface module 314 may also be used by a program administrator or a program provider to input one or more threshold metrics. The threshold metrics may be specific to a user or a group of users. In some examples, the threshold metrics may be the same for all users. In some examples, the threshold metrics may be provided as input to remote processing device 110 or remote cloud server 112 and communicated to processing device 108 using communication unit 304.

The user interface module 314 can be used by a healthcare practitioner to input healthcare assessment data related to a user. The healthcare assessment data may be data pertaining to a clinical assessment (e.g. performed by a doctor) of a user's health condition. For example, for a wearable insole device, the healthcare assessment data may relate to a user's DFU condition, as determined by a podiatrist. Alternatively, the healthcare assessment data may be monitoring data or monitoring metrics related to remote patient monitoring of a user. In some examples, the healthcare assessment data may be provided as input to the user interface module 314, the remote processing device 110 or remote cloud server 112 and communicated to the processing device 108 using communication unit 304.

Display 306 may be a LED or LCD based display and may be a touch sensitive user input device that supports gestures. Display 306 may be integrated into processing device 108. In some examples, display 306 may be located physically remote from processing device 108 and communicate with processing device 108 using communication unit 304. For example, display 306 may be provided as a handheld display to a user while processing device 108 may be integrated into the wearable device.

I/O unit 312 can include at least one of a mouse, a keyboard, a touch screen, a thumbwheel, a trackpad, a trackball, a card-reader, a foot-operated controller, voice recognition software and the like, depending on the particular implementation of processing device 108. In some cases, some of these components can be integrated with one another.

Power unit 316 can be any suitable power source that provides power to processing device 108 such as a power adaptor or a rechargeable battery pack depending on the implementation of processing device 108 as is known by those skilled in the art.

Memory unit 310 includes software code for implementing an operating system 320, programs 322, database 324, use metric module 326, monitoring metric module (not shown), user score module 328, alert module (not shown), threshold metric module 330, risk score module 332, model generation module 334, and model training module 336.

Memory unit 310 can include RAM, ROM, one or more hard drives, one or more flash drives or some other suitable data storage elements such as disk drives, etc. Memory unit 310 is used to store an operating system 320 and programs 322 as is commonly known by those skilled in the art. For instance, operating system 320 provides various basic operational processes for processing device 108. For example, the operating system 320 may be an operating system such as Windows® Server operating system, or Red Hat® Enterprise Linux (RHEL) operating system, or another operating system.

Database 324 may include a Structured Query Language (SQL) database such as PostgreSQL or MySQL or a not only SQL (NoSQL) database such as MongoDB, or Graph Databases, etc. Database 324 may be integrated with processing device 108. In some examples, database 324 may run independently on a database server in network communication with processing device 108.

Database 324 may store the use data received by processing device 108. Processing device 108 may receive use data generated by sensors 106 from electronics module 104. In some examples, database 324 may also store use data received from remote processing device 110 and/or cloud server 112. Database 324 may also store computed use metrics, threshold metrics and/or user scores. Database 324 may also store models generated by model generation module 334.

Programs 322 include various programs so that processing device 108 can perform various functions such as, but not limited to, receiving use data generated by sensors of a wearable device, generating monitoring data, computing use metrics, computing monitoring metrics, updating user scores, updating alerts and providing indications of use metrics, monitoring metrics, threshold metrics, alerts and user scores on a display device.

Use metric module 326 may compute one or more use metrics that indicate usage of the wearable device by the user. Use metric module 326 can compute use metrics in real-time based on use data collected by sensors 106 of the wearable device. In some examples, use metric module 326 can compute use metrics based on historical use data stored, for example, in database 324. Use metrics may be computed based on absolute use statistics, or may be computed based on relative use statistics.

In some examples, use metric module 326 can compute the use metrics using basic arithmetic functions. For example, the use data collected by sensors 106 may include data corresponding to each step taken by a user. Use metric module 326 can compute a step count use metric by adding all the steps taken by the user during a specified time period. Use metric module 326 can also compute an average step count use metric by performing averaging functions of the total step count over specified time periods.

In some examples, use metric module 326 can compute the use metrics using logical and/or arithmetic functions. For the example wearable insole device, use metric module 326 can determine a use status (“Yes” or “No”) of the wearable insole device based on the pressure and/or temperature data received from sensors 106 of the wearable insole device. Use metric module can compute a duration of use metric based on the determined use status over specified time periods.

In some examples, use metric module 326 can compute use metrics based on a combination of use data received from sensors 106 with other use data that provides further insight into use of the wearable device. The other use data can include interaction data of the user with a digital platform or app associated with the wearable device, data indicating responsiveness of the user to one or more alerts provided by the wearable device, usage data of a wireless charger for the wearable device, care personnel inputs, and the user's care level data for the wearable device. As an example, a digital app associated with the wearable device may provide an alert to a user regarding incorrect usage of the wearable device. Use metric module 326 can receive data related to the alert and sensor data indicating usage of the wearable device after the alert. Based on the received data, use metric module 326 can compute a response time metric indicating time taken by the user to correct usage of the wearable device after receiving the alert.

Use metric module 326 may provide a real-time indication of the computed use metrics to the user using user interface module 314. In some examples, use metric module 326 may store the computed use metrics in database 324. Use metric module can also provide the computed use metrics to other authorized users, remote processing device 110 or remote cloud server 112 using, for example, communication unit 304.

For an example wearable insole device, use metric module 326 may compute different use metrics indicating use of the wearable device by the user including, for example, a duration of wear use metric, a number of consecutive or non-consecutive days of use metric, a response time use metric to alerts provided by the wearable device, a response time use metric to notifications provided by the processing device, an activity level use metric, an interaction level use metric with a digital platform associated with the wearable device, a wireless charger use level metric, care personnel inputs, and a wearable device care level use metric.

User score module 328 can compare the use metrics computed by use metric module 326 to one or more threshold metrics. The threshold metrics may be provided by a program provider or program administrator and stored in database 324. In some examples, the threshold metric may be computed by threshold metric module 330.

There may be multiple threshold metrics associated with each use metric. For example, there may be four threshold metrics associated with a daily hours of use metric of a wearable insole device—a baseline threshold metric of 1.5 hours, a first improved outcome threshold metric of 3 hours, a second improved outcome threshold metric of 4.5 hours and a maximum threshold metric of 6 hours. Other use metrics may have multiple threshold metrics based on medical billing requirements. For example, there may be two threshold metrics associated with a non-consecutive days of use metric depending on the medical billing requirements for the wearable insole device: a first check-in threshold metric of 11 non-consecutive days of use at the 20^{th} day of the month, and an overall threshold metric of 16 non-consecutive days of use at the last day of the month.

User score module 328 can update a user score based on the results of the comparison of the use metric with the threshold metrics. The user score can indicate a user's consistency of use of the wearable device. For the example use metric of daily hours of use of the wearable insole device and an associated threshold metric of 4.5, the user score can be updated if the user uses the wearable insole for more than 4.5 hours in a day.

The updated user score can be used to provide positive incentives to the user encouraging the user to maintain proper and consistent use of the wearable device, and/or further improve the user's usage of the wearable device. For example, the user score may be used to provide monetary incentives that are in compliance with applicable laws and regulations. The monetary incentive can be based on the value of the user score. For example, a user score of 100 may correspond to a monetary incentive of $10. In some examples, other incentives like gifts, discounted goods or services, reduced insurance premiums, monetary donations (e.g., to charitable causes), loyalty program points, or digital game assets like skins, characters, levels etc. may be provided based on the value of the user score.

In some examples, user interface module 314 may generate a user interface such as a web page that enables a user to select which incentive to receive from among multiple options (e.g., a user may select to receive a $5 monetary incentive from among multiple options including the monetary incentive, a 50 loyalty program points incentive and a specific digital game asset incentive). The multiple options presented to the user may be based on the user score and/or rules set by the program provider or program administrator. In some examples, the web page may be generated by remote processing device 110 or remote cloud server 112 and user interface module 314 provides to the user a link to the web page. In other examples, the user interface may be a mobile app display, or an automated telephone call. In some examples, user interface module 314 may provide a phone number that a user can call to contact a program provider or program administrator that can help the user navigate the multiple options and select an incentive to receive.

The positive incentives can incentivize consistent or improved usage of the wearable device and result in improved efficiency of the wearable device in improving the user's health, fitness outcomes, and/or risk factors associated with a certain health outcome. After an incentive is provided to a user, user score module 328 can deduct the corresponding score amount from the user score.

In some examples, a single user score may be associated with all the use metrics of a user. In other examples, multiple user scores may be associated with a single user, for example, one user score for each of the user's multiple wearable devices.

User score module 328 can update the user score by different score amounts based on the comparison of the use metric with the threshold metrics. For example, user score module 328 may update the user score by one in response to the daily hours of use metric exceeding the associated improved outcome threshold metric for one day. In some examples, a linear or exponential relationship may be used for updating the user score. For example, user score module 328 may use a multiple of 0.1 to update the user score for each day that the daily hours of use metric exceeds the corresponding threshold metric. In this example, if the daily hours of use metric exceeds the corresponding threshold metric for 2 days, then user score module 328 updates the user score by 0.2 (multiplying 2 by 0.1). In another example, the user score module 328 may use a multiple of 0.1 to update the user score for each day that the non-consecutive days of use metric is on track to achieve the threshold metric of 16 non-consecutive days in a month (i.e. greater than 50% non-consecutive days of use). In this example, if the user has 6 non-consecutive days of use at the 8^{th} day in the month ( 6/8=75% non-consecutive days of use), and the user has remained above the 50% pro-rated threshold metric for the 8 days, then the user score module 328 updates the user score by 0.8 (multiplying 8 by 0.1). Alternatively, the user score may be updated for each non-consecutive day of use achieved out of the goal of 16 non-consecutive days in a month. In this example, if the user has 6 non-consecutive days of use at the 8^{th} day in the month, the user score module 328 updates the user score by 0.6 (multiplying 6 by 0.1). Every additional non-consecutive day of use recorded will result in an additional 0.1, until the threshold metric is achieved, or the end of the month is reached.

In some examples, user score module 328 may update the user score based on a value of the use metric. For example, user score module 328 may update the user score by seven in response to a daily hours of use metric of seven for that day. In some examples, user score module 328 may use a linear or exponential relationship between the use score metric and the update to the user score. For example, user score module 328 may update the user score by seventy in response to a daily hours of use metric of seven for that day.

To prevent overuse of the wearable device, user score module 328 may not update the user score by additional score amounts after the maximum threshold metric is exceeded. For an example maximum threshold metric of ten hours of daily use and a daily hours of use metric of twelve hours on a given day, user score module 328 may only update the user score by ten (corresponding to the maximum threshold of ten hours) instead of twelve (corresponding to the use metric of twelve hours). In some examples, to discourage overuse of the wearable device, user score module 328 may not update the user score—or the score may be decreased—for a given day in response to the use metric exceeding the maximum threshold metric.

User score module 328 can provide a real-time indication of user scores and a score amount by which the user score was recently updated using user interface module 314.

In response to the use metric exceeding a threshold metric, user score module 328 can compute a potential score amount update corresponding to the use metric exceeding the next incremental threshold metric. For example, in response to the daily hours of use metric exceeding a first associated improved outcome threshold metric of three hours, user score module 328 can compute a potential score amount update corresponding to the use metric exceeding a second associated improved outcome threshold metric of six hours. User score module 328 can provide an indication to the user of the next incremental threshold metric and the potential score amount update for achieving the next incremental threshold metric. This may motivate the user to achieve incremental improvements in the use metric and thereby improve the efficiency of the wearable device in improving health and/or fitness outcomes.

In some embodiments, user score module 328 can compare the use metrics computed by use metric module 326 to one or more challenge metrics (in addition to the threshold metrics). The challenge metric may be provided by a program provider or program administrator. In some examples, the challenge metric may be provided in response to a user health assessment or user risk score indicating a high risk of disengagement before reaching the next threshold metric. For example, a user may have achieved a baseline threshold metric of two hours of daily usage for two consecutive weeks but the user may not have achieved an improved outcome threshold metric of six hours of daily usage on any of the days. A challenge metric of three hours may be provided to incentivize usage and reduce risk of disengagement of the user. A challenge score amount may be associated with the challenge metric indicating the amount by which the user score can be updated for achieving the challenge metric.

The challenge metric may be activated in real-time. For example, user score module 328 may begin comparing the use metric to the challenge metric immediately after the challenge metric is received at processing device 108. In some examples, the received challenge metric may be stored in database 324 and scheduled for future activation. For example, the challenge metric may be scheduled to be active for the next week of usage of the wearable device.

In response to receiving a challenge metric, processing device 108 can provide an indication to the user (e.g., on display 306) regarding the challenge metric and the associated challenge score amount.

User score module 328 can update the user score based on the results of the comparison of the use metric with the challenge metric. For example, in response to the use metric exceeding the challenge metric, user score module 328 can update the user score by the challenge score amount. User score module 328 may also provide, to the user, a notification (e.g., a congratulatory message) and an indication regarding the challenge score amount achieved and the updated user score.

A monitoring metric module (not shown) may compute one or more monitoring metrics that indicate the level or quality of monitoring of use of the wearable device. The monitoring metric module can compute monitoring metrics in real-time based on monitoring data collected by the processor unit 308, in communication with the wearable device. The monitoring data may be generated when a monitoring action is performed (e.g. when an additional user reviews sensor data of the user). In some examples, the monitoring metric module can compute monitoring metrics based on historical monitoring data stored, for example, in database 324.

In some examples, the monitoring metric module can compute the monitoring metrics using basic arithmetic functions. For example, the monitoring data collected by the processor unit 308 may include data corresponding to remote patient monitoring time (i.e. time spent reviewing sensor data, use data, or use metrics). The monitoring metric module can compute the remote patient monitoring time by adding all the minutes spent using the user interface during a specified time period. The monitoring metric module can also compute an average remote patient monitoring time metric by performing averaging functions of the total remote patient monitoring time over specified time periods.

In some examples, the monitoring metric module can compute the monitoring metrics using logical and/or arithmetic functions. For the example wearable insole device, the monitoring metric module can determine a monitoring use status (“Yes” or “No”) of the user interface. The monitoring metric module can compute a duration of monitoring metric based on the determined monitoring use status over specified time periods.

The monitoring metric module may provide a real-time indication of the computed monitoring metrics to the user and/or healthcare practitioner using user interface module 314. In some examples, the monitoring metric module may store the computed monitoring metrics in database 324. The monitoring metric module can also provide the computed monitoring metrics to other authorized users, the remote processing device 110 or the remote cloud server 112 using, for example, the communication unit 304.

For an example wearable insole device, the monitoring metric module may compute different monitoring metrics for a user using the wearable device including, for example, remote patient monitoring time totals (i.e. time spent reviewing sensor data, use data, or use metrics) and number of practitioner-user interactions in a time period.

An alert module (not shown) may compare the use metrics computed by use metric module 326 or monitoring metrics computed by the monitoring metric module to one or more threshold metrics. The threshold metric may be provided by a program provider or program administrator and stored in database 324. In some examples, the threshold metric may be computed by threshold metric module 330.

The alert module can generate an alert based on the results of the comparison of the use metric or monitoring metric with the threshold metric. The alert module can indicate a user's quality or consistency of interaction with a healthcare practitioner or monitoring device. For the example monitoring metric of monthly call interaction time and an associated threshold metric of 20 minutes, an alert may be generated if 20 minutes is not reached by the 20^{th} day of the month. Use metrics or monitoring metrics used by the alert module can indicate a lack of consistency in use and monitoring and generate an alert. In another example, the monitoring device may generate an alert when the user has not worn the insoles for more than 0.9 hours or greater than 0 steps for more than 3 consecutive days. In the example, the lack of use data or sensor data may be used to initiate the alert. In a third example, the monitoring device may generate an alert when a user with a wearable insole device containing temperature sensors has excessive temperature flags at the same contralateral locations on their feet. The excessive alerts may be recognized as a natural temperature offset between the user's feet and actions may be initiated to improve the temperature flag detection accuracy for the user. The alert module may also generate alerts for any trends in monitoring metrics affecting medical billing requirements.

In some examples, the alert module may generate a user interface such as a web page that enables a user, program provider, program administrator or healthcare practitioner to review and alter the generated alerts. In some examples, the web page may be generated by remote processing device 110 or remote cloud server 112 and user interface module 314 may provide a link to the web page. In other examples, the user interface may be a mobile app display, or an automated telephone call. The alert module may generate the user interface for a single user or multiple users (i.e. one healthcare practitioner has a user interface to monitor multiple wearable device users). The alert module can provide a real-time indication of generated alerts using user interface module 314.

In some examples, a single alert may be associated with all the monitoring metrics or use metrics of a user. In other examples, multiple alerts may be associated with a single user, for example, one alert for each of the user's multiple wearable devices.

A reporting module (not shown) may also compare the use metrics computed by use metric module 326 or monitoring metrics computed by the monitoring metric module to one or more threshold metrics. The threshold metric may be provided by a program provider or program administrator and stored in database 324. In some examples, the threshold metric may be computed by threshold metric module 330.

The reporting module can generate a report based on the results of the comparison of the use metric or monitoring metric with the threshold metric. The reporting module may also complete various data analytics and reporting methods on use metrics, monitoring metrics, alerts, threshold metrics and/or user scores. The reporting module can report data trends and graphics indicating a user's quality or consistency of interaction with a healthcare practitioner or monitoring device. The report may be generated at regular time intervals (e.g. monthly or daily), when triggered (e.g. when a monitoring metric satisfies a threshold requirement), or when requested (e.g. a healthcare practitioner requests to review the report in real-time).

In some examples, the reporting module may generate a user interface such as a web page that enables a user, program provider, program administrator or healthcare practitioner to review and alter the generated reports. In some examples, the web page may be generated by remote processing device 110 or remote cloud server 112 and user interface module 314 may provide a link to the web page. In other examples, the user interface may be a mobile app display, or an automated telephone call.

In some examples, the reporting module may generate reports via data analytics and reporting software (e.g. Power BI® software owned by Microsoft Corporation of Washington, USA). The data analytics and reporting software may issue the reports via the user interface. The user interface may also provide a link to review and alter generated reports on the data analytics and reporting application (e.g. Power BI® dashboard). The reports may be reviewed by the healthcare practitioner, program provider, program administrator, user, or monitoring device. The statistics and trends in the reports may be used to initiate actions to improve consistency of use of the wearable device or remote patient monitoring. For example, a healthcare practitioner may review a report on the use metric of non-consecutive days of use, and the report may show that the user has not satisfied the threshold requirement of exceeding the threshold metric of 16 non-consecutive use days in the month. In the example, the healthcare practitioner may be prompted to increase the monthly RPM time total for the given user. The reporting module may generate the user interface for a single user or multiple users (i.e. one healthcare practitioner has a user interface to monitor multiple wearable device users).

Model generation module 334 may implement one or more machine learning models, for example, a risk score model and a threshold metric model. The machine learning models may include, for example, a logistic regression model, a polymeric regression model, a neural network, a decision tree, a support vector machine or an adaboost decision tree. The generated models may be stored in database 324.

Risk score module 332 may use the risk score model to determine a risk score indicating risk of disengagement of the user from using the wearable device. Threshold metric module 330 may use the threshold metric model to generate and/or determine updates to the threshold metrics.

The risk score model can be configured to receive use metrics, user demographic data, monitoring metrics, and/or additional user data as inputs and generate a risk score as output. The use metrics may correspond to a specific period of use of the wearable device. For example, the use metrics may correspond to the previous 60 days of use of the wearable device. In other examples, periods longer or shorter than 60 days may be used.

The risk score model may also generate a confidence score associated with the risk score output. In some examples, model training module 336 may retrain the risk score model when the confidence score associated with the risk score output falls below a threshold confidence score.

For the example wearable insole device, the use metrics inputs may include, for example, duration of wear of the wearable device by the user, a number of consecutive or non-consecutive days of use of the wearable device by the user, a duration of wear of the wearable device by the user within a predetermined time interval, a response of the user to one or more alerts provided by the wearable device, a response of the user to one or more indications provided by the processor, an activity level of the user, an interaction level of the user with a digital platform associated with the wearable device, a use level of a wireless charger for the wearable device by the user, care personnel inputs (e.g., in-person or remote), and a care level of the wearable device by the user. The use metric inputs can be provided, for example, by use metric module 326.

The user demographic data input can include, for example, age, gender, or location of the user. The user demographic data may be provided by the user using user interface module 314. In some examples, the user demographic data may be provided by a program provider or a program administrator via communication unit 304.

The monitoring metrics can include, for example, monthly remote patient monitoring time and monthly number of practitioner-user interactions. The monitoring metric inputs can be provided, for example, by the monitoring metric module.

The additional user data input may include data provided by the user in response to a questionnaire. In some examples, the additional user data may include the user's health assessment data provided by a healthcare practitioner, a program provider or a program administrator. For example, if a user's health assessment indicates an improvement in the user's DFU condition after consistent usage of the wearable device for a time period, the risk score of the user being disengaged from using the wearable device may be reduced.

The risk score output of the risk score model may include a classification of the user. For example, the risk score output may include a classification of the user as “consistent” or “disengaged”. In some examples, the risk score output may include a numerical value. For example, the risk score output may include a probability value indicating risk of disengagement of the user from using the wearable device.

The threshold metric model can be configured to receive use metrics, monitoring metrics, risk score indicating risk of disengagement from usage of the wearable device and/or current threshold metrics for the user and generate a new or updated threshold metric as output. The use metrics may correspond to a specific period of use of the wearable device. For example, the use metrics may correspond to the previous 60 days of use of the wearable device. In other examples, periods longer or shorter than 60 days may be used. The risk score indicating risk of disengagement from usage of the wearable device may be provided by risk score module 332 using the risk score model generated by model generation module 334.

For the example wearable insole device, the use metrics inputs may include, for example, duration of wear of the wearable device by the user, a number of consecutive or non-consecutive days of use of the wearable device by the user, a duration of wear of the wearable device by the user within a predetermined time interval, a response of the user to one or more alerts provided by the wearable device, a response of the user to one or more indications provided by the processor, an activity level of the user, an interaction level of the user with a digital platform associated with the wearable device, a use level of a wireless charger for the wearable device by the user, and a care level of the wearable device by the user. The use metric inputs can be provided, for example, by use metric module 326.

The monitoring metrics can include, for example, monthly remote patient monitoring time total and monthly number of practitioner-user interactions. The monitoring metric inputs can be provided, for example, by the monitoring metric module.

In some examples, model generation module 334 may be located at remote processing device 110 or remote cloud server 112. Model generation module 334 may generate risk score models and/or threshold metric models based on input data for multiple users. Model generation module 334 may generate the models based on input data including use metrics, user demographic data, monitoring metrics, additional user data and/or current threshold metrics for multiple users. In some examples, groups of users may be created, for example, groups based on user demographic information. Model generation module 334 may generate different models for different groups of users based on corresponding input data for that group of users. Model generation module 334 can then provide the generated models to processing device 108 of the different users using communication unit 304.

Model training module 336 may train the models generated by model generation module 334. Although shown separately, model generation module 334 and model training module 336 may be implemented together as a combined model generation module.

Model training module 336 can train the risk score model and/or the threshold metric model using training data that includes a set of inputs, for example, use metrics, user demographic data, monitoring metrics, additional user data, and/or threshold metrics for a time period. The training data can also include a set of corresponding outputs, for example, risk score classifications of “consistent” or “disengaged” for one or more users during that time period or a risk score probability determined based on a number of users from a group of users being disengaged from using the wearable device during that time period.

In some examples, model training module 336 may use training data corresponding to a single user to specifically train the models for that user. For example, the risk score model may be trained to generate risk score output for a specific user. In some examples, model training module may use training data for multiple users or a group of users to train corresponding models for the multiple users or specific groups of users.

Model training module 336 may perform an initial training of the models generated by model generation module 334. Threshold metric module 330 and/or risk score module 332 may begin using the generated models after they are trained using the training data. Further, model training module 336 may retrain the models at regular time intervals or when an input requesting retraining is received (e.g., from a program provider or a program administrator). In some examples, model training module 336 may retrain the risk score model when the confidence score associated with the risk score output falls below a threshold confidence score.

In some examples, model training module 336 may retrain the models based on a mismatch between observed data and predicted data. For example, the risk score model may be retrained if multiple users are disengaged from using the wearable device even while the risk score model predicted a low risk of disengagement for those users.

The training process may vary depending on the type of machine learning model being implemented. For example, with a regression model, an optimization algorithm can be applied to optimize the regression coefficients implemented by the model. The optimization algorithm may employ a cost function based on the difference between the desired outputs and the model outputs.

Risk score module 332 can determine a risk score indicating risk of disengagement of a user from using the wearable device. Risk score module 332 can determine the risk score using the risk score model generated by model generation module 334 and trained by model training module 336.

Risk score module 332 may determine an initial risk score for a new user. For example, risk score module 332 may provide demographic data and/or additional user data for the new user as inputs to the risk score model. Risk score module 332 may provide the generated risk score output to threshold metric module 330 to generate or determine updates to the threshold metrics for the new user. Risk score module 332 may also store the generated risk score output in database 324. In some examples, if the risk score output indicates a high risk of disengagement of the user, risk score module 332 may provide a notification to a program provider or program administrator. The program provider or program administrator may take specific actions (e.g., provide additional incentives or challenge metrics) to incentivize usage of the wearable device and thereby increase the efficiency of the wearable device in improving health and/or fitness outcomes. Additionally, an increase in frequency of practitioner-user interactions may be provided to increase engagement and encourage continuous and proper use of the wearable device. In some examples, if the risk score output indicates a high risk of disengagement of the user, risk score module 332 may generate an additional challenge. For examples, the risk score module 332 may notify the user that if they achieve an additional 2000 steps within a predetermined period above their typical baseline, they will be rewarded with a bonus score.

Risk score module 332 may also determine additional risk scores for the user at regular time intervals. The parameter specifying the time interval may be stored in database 324 and can be customized for each user. In some examples, risk score module 332 may determine the additional risk scores at more frequent time intervals (e.g., two times the frequency of the regular time interval) if one or more previous risk scores for the user indicate a high risk of disengagement.

Risk score module 332 may also determine additional risk scores based on the values of the use metrics or the monitoring metrics. Risk score module 332 may determine the additional risk scores based on either the absolute value of the use metrics or a rate of change in the values of the use metrics or the monitoring metrics. For example, risk score module 332 may determine additional risk scores in response to the daily hours of use metric dropping below a lower threshold value. As another example, risk score module 332 may determine additional risk scores in response to a large percentage drop (greater than a higher threshold percentage value) in the daily hours of use metric over a one-week period. In some examples, risk score module 332 may determine an additional risk score each time a new use metric is computed (e.g., each time a weekly hours of use metric is computed).

In some examples, risk score module 332 may determine additional risk scores in response to inputs provided by a program provider, a program administrator or a healthcare practitioner. For example, risk score module 332 may determine an additional risk score either automatically or in response to an input provided by a program provider, administrator or healthcare practitioner at remote processing device 110.

Threshold metric module 330 may generate one or more threshold metrics using the threshold metric model generated by model generation module 334 and trained by model training module 336. For example, threshold metric module 330 may generate the initial threshold metrics for a new user of the wearable device. In some examples, the initial threshold metrics for a new user of the wearable device may be provided by a program provider, a program administrator or a healthcare practitioner. For example, threshold metrics may be provided based on monitoring metrics of a healthcare practitioner providing remote patient monitoring to a user.

Threshold metric module 330 can provide to the threshold metric model a risk score input for the new user generated by risk score module 332. In some examples, threshold metric module 330 can also provide initial threshold metrics provided by a program provider, a program administrator or a healthcare practitioner as input to the threshold metric model. Threshold metric module 330 can generate customized threshold metrics (tailored to the specific disengagement risk score) for the new user based on the output of the threshold metric model.

Threshold metric module 330 may determine updates to the threshold metrics using the threshold metric model generated by model generation module 334 and trained by model training module 336. For example, threshold metric module 330 may determine the updates to the threshold metrics at regular time intervals. The parameter specifying the time interval may be stored in database 324 and can be customized for each user. For an example use metric of an activity level of a user, threshold metric module 330 may provide as input to the threshold metric model the activity level use metrics and corresponding threshold metrics for a period of sixty days. In some examples, threshold metric module 330 may determine updates to the threshold metrics based on the history of use metrics, monitoring metrics, use scores, and/or alerts over the previous time interval. The threshold metric model can determine updates to the threshold metrics based on an impact of previous changes in the threshold metric on the activity level use metric. In some examples, threshold metric module 330 may determine updates to the threshold metrics at more frequent time intervals (e.g., three times the frequency of the regular time interval) if one or more previous risk scores for the user indicate a high risk of disengagement.

Threshold metric module 330 may also determine updates to the threshold metrics each time risk score module 332 determines a new risk score for the user. In some examples, threshold metric module 330 may only determine updates to the threshold metrics in response to a new risk score that is higher than a threshold risk score.

In some examples, threshold metric module 330 may determine updates to the threshold metrics each time use metric module 326 computes new use metrics for the user. In some examples, threshold metric module 330 may only determine updates to the threshold metrics in response to the new use metrics falling below a threshold value. The threshold values may be calibrated based on, for example, medical research data. For an example daily hours of use metric of a wearable insole device, a calibrated threshold value may be five hours to prevent DFU development. In response to the daily hours of use metric for a user falling from six hours to four hours, threshold metric module 330 may determine an update to a threshold metric to provide a positive incentive to the user to improve usage of the wearable insole device from four hours to a value greater than five hours. This can improve the effectiveness of the wearable insole device in preventing DFU development. In some examples, threshold metric module 330 may determine updates to the threshold metrics each time monitoring metric module computes new monitoring metrics for the user or each time the alert module generates a new alert.

In some examples, threshold metric module 330 may determine updates to the threshold metrics in response to inputs provided by a program provider, a program administrator or a healthcare practitioner. For example, threshold metric module 330 may determine updates to the threshold metrics in response to an input provided by a program administrator at remote processing device 110.

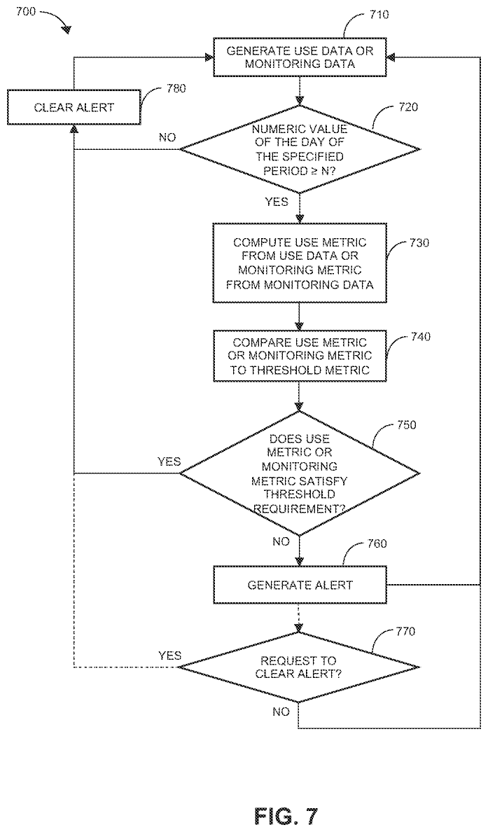

Referring now to FIG. 7, shown therein is a flowchart illustrating an example of a method 700 for analyzing data generated by use of a wearable device by a user. Method 700 can be implemented, for example, by system 100 shown in FIG. 1 and/or system 300 shown in FIG. 3.

Method 700 may be performed regularly, such as daily or hourly. At 710, use data or monitoring data may be generated. Use data may be generated from sensors of a wearable device. For example, a user may begin using a wearable insole device and processing device 108 may receive the use data generated from sensors 106. In some examples, the use data may be stored in database 324. Alternatively, monitoring data may be generated by a monitoring device (not shown). For example, the monitoring device can include the remote processing device 110 or the cloud server 112 in FIG. 1 or the processor unit 308, the user interface module 314, or the display 306 in FIG. 3. In some examples, the monitoring device includes the monitoring metric module and/or alert module. Monitoring data may be generated when a monitoring action is performed (e.g. when an additional user reviews sensor data, use data, or use metrics of the user of the wearable device), using the monitoring device. In some examples, the monitoring data may be stored in database 324.

At step 720, a numeric value of a day of a specified period is compared to a numeric value of a predefined day of the specified period, N. The alert module (not shown) checks if the numeric value of the day of the specified period is greater, equal to, or less than N. The specified period may be any length of time comprising at least one day, such as one day, two weeks, one month, or one year. For example, N may be 20 and the specified period may be a month. If the numeric value of the day of the month is not greater than 20 (e.g. if the day of the month is the 10^{th}, which is less than the 20^{th} ), the alert module proceeds to step 780. At step 780, alerts are cleared (such as alerts from previous months), and method 700 repeats from step 710. If the numeric value of the day of the month is greater than 20 (e.g. if the day of the month is the 27^{th}, which is greater than the 20^{th}), the system proceeds to step 730.

At step 730, a use metric is computed from the use data, or a monitoring metric is computed from the monitoring data. For example, wherein the monitoring data is the time the additional user spends on a screen in the user interface device 314, the monitoring metric may be a monthly remote patient monitoring time total for a given user (i.e. the time the additional user spends reviewing the user's use data and use metrics).

The monitoring metric computed at step 730 may then be compared to a threshold metric at step 740. The monitoring metric may be computed by the monitoring metric module and the threshold metric may be computed by the threshold metric module 330.

At 750, the alert module determines whether the use metric or the monitoring metric satisfies a threshold requirement (i.e. is above or below the threshold metric). If the use metric or the monitoring metric does satisfy the threshold requirement, the alert module proceeds to 780 and clears alerts (e.g. alerts that may have been presented on an earlier date, when the threshold requirement was not satisfied). If the use metric or the monitoring metric does not satisfy the threshold requirement, an alert is generated at 760. The alert may be generated by the monitoring device and presented to the user or to the additional user, for example, on the user interface module 314. The alert may also be passed on to a program provider or program administrator. The method then returns to step 710, and method 700 repeats. The alert generated at step 760 will not be cleared unless a new specified time period begins (i.e. at 720 the numeric value of the day of the specified time period returns to 1, which will typically be less than N), or if the threshold requirement is met at 750 when method 700 repeats.

Optionally, an alert may also be cleared at 770, if the user or the additional user requests to clear the alert. For example, the user, healthcare practitioner or monitoring device may assess the alert and then designate the alert as non-useful, inaccurate, or already addressed by entering a request to clear the alert or pressing a button to clear the alert. The request to clear the alert can be initiated, for example, through the user interface module 314.

Three embodiments of method 700 will now be described with reference to remote patient monitoring. In all embodiments, the additional user is a healthcare practitioner that monitors sensor data, use data, and use metrics of wearable device users through an online dashboard, which may be, for example, the display 306 or the user interface module 314.

In a first embodiment, monitoring data is generated at 710, and the monitoring data is the time the healthcare practitioner spends on a screen in the online dashboard, which is automatically tracked by the online dashboard. At 720, the alert module checks the numeric value of the day of the month and compares it to N, which is 20 (i.e. the 20^{th} day of the month). If the date is after the 20^{th} of the month (e.g. the 30^{th}), at 730, a monitoring metric is computed. In this example, the monitoring metric is monthly remote patient monitoring (RPM) time. The monthly RPM time total is the time spent by the healthcare practitioner in reviewing and following up on data collected from a given user's wearable device in a one month period. For example, reviewing sensor data and use data, investigating concerns in sensor data, use data, or use metrics, and interacting with a given user (e.g. over a telephone call) may all be activities performed by a healthcare practitioner that contribute towards the monthly RPM time total. At 740, the monthly RPM time total is compared to a threshold metric. The threshold metric (i.e. the threshold monthly RPM time total) may be 20 minutes. At 750, it is determined whether the monthly RPM time total meets or exceed the 20 minute threshold metric. If the monthly RPM time total is below this threshold, an alert may be generated on the online dashboard at 760. The alert may be presented in the form a visual notification, such as an alarm clock icon. The alert may prompt the healthcare practitioner to increase the monthly RPM time total for the given user. The alert may be cleared when the method 700 repeats on a subsequent day, if the healthcare practitioner has increased the monthly RPM time total to surpass the 20 minute threshold. For example, if the monthly RPM time total is 15 minutes on the 20^{th} of day of the month, then increases to 21 minutes on the 21^{st} day of the month, the alert will clear on day 21. Alternatively, the alert may clear at the start of a new month (since at 720 the 1^{st} day of the month is less than the 20^{th}), or if the healthcare practitioner requests to clear the alert,

In a second embodiment, monitoring data is generated at 710, and the monitoring data is the number of interactions between the healthcare practitioner and the user (e.g. appointment activity, follow-up activity, patient education activity). The interactions may be recorded directly by the online dashboard, if the online dashboard has communication features (e.g. emailing or calling services) set up within the dashboard. Alternatively, the healthcare practitioner may manually enter interactions into the online dashboard (e.g. if the healthcare practitioner calls a user on their cellphone, they can manually record the date/time of the interaction in the online dashboard). At 720, if the numeric value of the day of the month is greater than 20 (i.e. the 20^{th} day of the month), step 730 is performed and a monitoring metric is calculated from the monitoring data. In this example, the monitoring metric is the monthly number of practitioner-user interactions. At 740 and 750, it may be determined whether the monthly number of practitioner-user interactions exceeds a threshold of 1 interaction. If this threshold is met or exceeded, any previous alerts are cleared at 780. If the threshold has not been met, an alert is generated on the online dashboard at 760. The alert may be presented in the form of a visual notification, such as a telephone icon. The alert may prompt the healthcare practitioner to increase their interactions with the user. The alert may be cleared when the method 700 repeats on a subsequent day, if the healthcare practitioner has increased their interactions with the user to surpass the 1 interaction requirement. For example, if the monthly number of practitioner-user interactions is 0 on the 25^{th} day of the month, and increases to 2 on the 26^{th} day of the month, the alert will clear on day 26. Alternatively, the alert may clear at the start of a new month (since at 720 the 1^{st} of a month is less than the 20^{th}), or if the healthcare practitioner requests to clear the alert,

In a third embodiment, use data is generated at 710, and the use data is the consecutive or non-consecutive days of use of the wearable insole device. A day of use may be considered any day where the wearable insole device records a step count greater than 0, or a wear time greater then 0.9 hours (54 minutes). In this embodiment, N is again 20 and the specified period is one month. The numeric value of the day of the month is compared to N at 720. At 730, the use metric is computed, which is the monthly number of non-consecutive days of use (i.e. total days of use over the month). At 740, the monthly number of non-consecutive days of use is compared to a threshold number of non-consecutive days of use. This threshold may be designated by a healthcare practitioner, a program provider or a program administrator. Alternatively, the threshold metric may be determined by a mathematic relationship to an overall goal for a longer period of time. For example, an overall goal of 16 non-consecutive days of use in a month (i.e. approximately 16/30=53% non-consecutive days of use) may be used to determine a pro-rated ratio of 11 non-consecutive days of use by the 20^{th} day (i.e. N) of the month ( 11/20=55% non-consecutive days of use), which is the closest full number of days that surpasses a ratio of 53%. The threshold metric may also be variable, depending on the day that method 700 is run. For example, if method 700 is run on day 20, the threshold metric will be 11, but if method 700 is run on day 25, the threshold metric will be 13 ( 13/25=56% non-consecutive days of use), the closest full number of days that surpasses a ratio of 53%. If at 750, it is determined that the monthly number of days of use is less than the number of days of use required to produce a ratio that surpasses 53%, an alert will be generated on the online dashboard and presented to the healthcare practitioner at 760. The alert may be presented in the form of a visual notification, such as a calendar icon. The alert may prompt the healthcare practitioner to intervene and assist the user in improving their number of non-consecutive days of use before the end of the period of time. The alert may also prompt the initiation of a foot self-assessment to ensure the lack of consistent use has not affected the user's foot health. For example, the healthcare practitioner may pass the alert along to the user via a telephone call or a digital display alert. In another example, the healthcare practitioner may review or alter the user's disengagement risk score. The healthcare practitioner may then increase contact with the user and/or implement other methods to reduce the user's disengagement risk score. The alert may be cleared when the user's number of non-consecutive days of use satisfies the threshold requirement. Alternatively, the alert may clear at the start of a new month (since at 720 the 1^{st} is less than the 20^{th}), or if the healthcare practitioner requests to clear the alert.

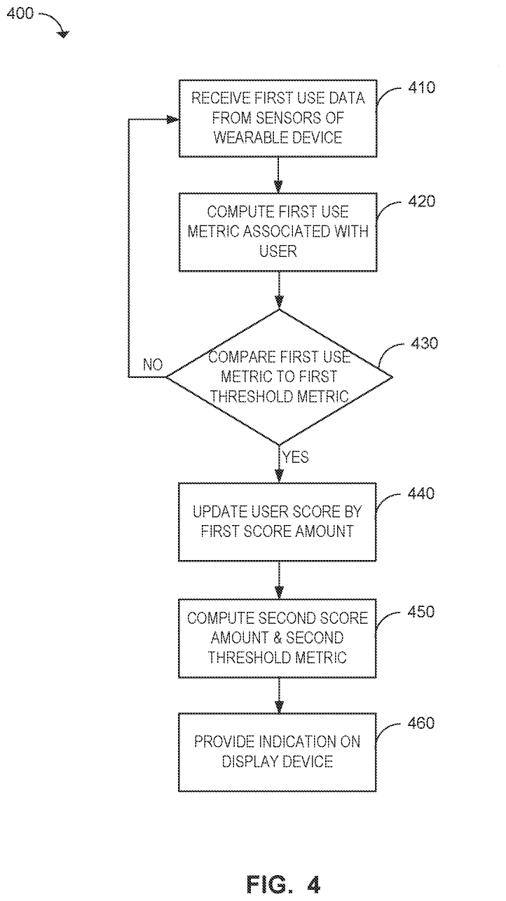

Referring now to FIG. 4, shown therein is a flowchart illustrating an example of a method 400 for analyzing data generated by use of a wearable device by a user. Method 400 can be implemented, for example, by system 100 shown in FIG. 1.

Method 400 may be performed at various times relating to use of the wearable device by the user. For example, method 400 may be performed each time the wearable device is in use for a specific duration of time, for example, ten minutes. In some examples, method 400 may be performed at regular time intervals independent of the usage of the wearable device, for example, every hour. Method 400 may also be performed in response to an input received from the user, a program provider or a program administrator.

At 410, use data may be received from sensors of a wearable device. For example, a user may begin using a wearable insole device and processing device 108 may receive use data from sensors 106. In some examples, the received use data may be stored in database 324.

At 420, a first use metric associated with the user may be computed based on the received use data. For example, use metric module 326 may compute a daily hours of use metric of the user for the wearable insole device. Use metric module 326 may compute the first use metric in real-time based on the received use data. In some examples, use metric module 326 may compute the use metric for specific periods of time based on the use data stored in database 324.

At 430, the first use metric may be compared with a first threshold metric. For example, user score module 328 may compare the first use metric with a first improved outcome threshold metric. The first improved outcome threshold metric can be a threshold metric generated by threshold metric module 330, or a threshold metric provided by a program administrator or program provider.

If the first use metric does not meet the comparison criterion, method 400 may continue to 410. If the first use metric meets the comparison criterion, method 400 may continue to 440. For example, if the first use metric is three hours of use of the wearable insole device and the first threshold metric is four hours of use of the wearable insole device, then the comparison criterion is not met and method 400 may continue to 410. If the first use metric is five hours, the comparison criterion is met and method 400 may continue to 440.

At 440, a user score may be updated by a first score amount. For example, user score module 328 may update a user score by a first score amount in response to the first use metric meeting the comparison criterion. The first score amount may be a fixed amount, for example, user score module 328 may increase the user score by a first score amount of 0.1 for each day that the first use metric exceeds the first threshold metric. In some examples, the first score amount may be based on values of the first use metric and/or the first threshold metric. For example, user score module 328 may increase the user score by a first score amount of five in response to a first use metric of five hours of use of the wearable insole device. Alternatively, user score module 328 may increase the user score by a first score amount of two in response to the first use metric of five hours exceeding the first threshold metric of three hours by two hours.

At 450, a second score amount and a second threshold metric may be computed. For example, threshold metric module 330 may compute the second threshold metric using the threshold metric model. The second threshold metric may be computed based on the use metrics for the user, the first threshold metric and/or a risk score for the user indicating risk of disengagement from usage of the wearable device. For example, if the first threshold metric is three hours of use of the wearable device and the risk score indicates low risk of disengagement of the user, threshold metric module 330 may compute the second threshold metric as six hours. But, if the risk score indicates high risk of disengagement of the user, threshold metric module 330 may compute the second threshold metric as five hours to incentivize further use of the wearable device. In some examples, the second threshold metric may need to be approved by a program provider or a program administrator before it becomes active and is used for subsequent comparisons. User score module 328 may determine a second score amount corresponding to the score amount by which the user score will be updated in response to the use metric meeting the second threshold metric.

At 460, a first indication of the user score, the second threshold metric and the second score amount may be provided to the user. For example, the first indication may be provided to the user on display 306 using user interface module 314. Based on the value of the user score, the user interface may enable the user to redeem a portion of the user score for incentives as described herein above. The user interface may also provide the user with information regarding the next incremental goal (the second threshold metric) and the corresponding incentive for achieving the next incremental goal (the second score amount).

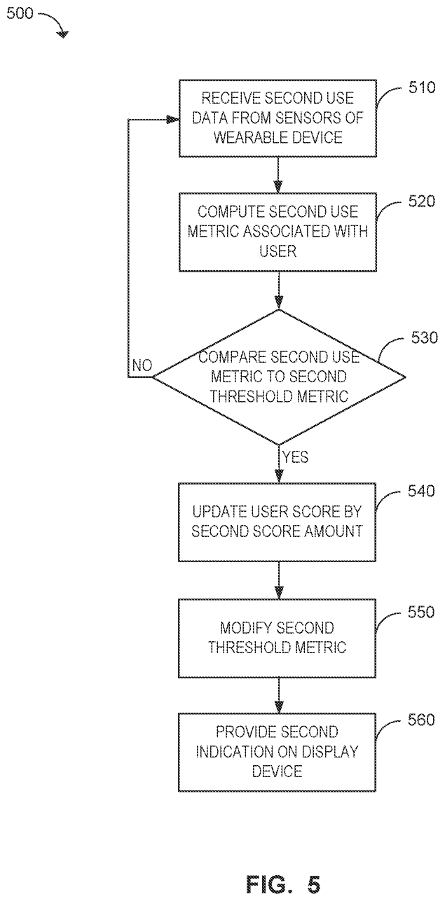

Referring now to FIG. 5, shown therein is a flowchart illustrating an example of a method 500 for analyzing data generated by use of a wearable device by a user. Method 500 can be implemented, for example, by system 100 shown in FIG. 1.

Method 500 may be performed at various times relating to use of the wearable device by the user. Method 500 may be operated independently. For example, method 500 may be performed each time the wearable device is in use for a specific duration of time or at regular time intervals independent of the usage of the wearable device. Method 500 may also be performed in response to an input received from the user, a program provider or a program administrator. In some examples, method 500 may be performed in conjunction with method 400 described herein above and may be performed subsequent to 460 of method 400.

At 510, second use data may be received from sensors of a wearable device. For example, a user may begin or continue using a wearable insole device and processing device 108 may receive use data from sensors 106. In some examples, the received use data may be stored in database 324.

At 520, a second use metric associated with the user may be computed based on the received use data. For example, use metric module 326 may compute a daily hours of use metric of the user for the wearable insole device. Use metric module 326 may compute the second use metric in real-time based on the received use data. In some examples, use metric module 326 may compute the second use metric for specific periods of time based on the use data stored in database 324.

At 530, the second use metric may be compared with a second threshold metric. For example, user score module 328 may compare the second use metric with a second improved outcome threshold metric. The second improved outcome threshold metric can be a threshold metric generated by threshold metric module 330, or a threshold metric provided by a program administrator or program provider.

If the second use metric does not meet the comparison criterion, method 500 may continue to 510. If the second use metric meets the comparison criterion, method 500 may continue to 540. For example, if the second use metric is five hours of use of the wearable insole device and the second threshold metric is six hours of use of the wearable insole device, then the comparison criterion is not met and method 500 may continue to 510. If the second use metric is seven hours, the comparison criterion is met and method 500 may continue to 540.

At 540, a user score may be updated by a second score amount. For example, user score module 328 may update a user score by a second score amount in response to the second use metric meeting the comparison criterion. The second score amount may be a fixed amount, for example, user score module 328 may increase the user score by a second score amount of 0.1 for each day that the second use metric exceeds the second threshold metric. In some examples, the second score amount may be based on values of the second use metric and/or the second threshold metric. For example, user score module 328 may increase the user score by a second score amount of seven in response to a second use metric of seven hours of use of the wearable insole device. Alternatively, user score module 328 may increase the user score by a second score amount of one in response to the second use metric of seven hours exceeding the second threshold metric of six hours by one hour.

At 550, the second threshold metric may be modified. For example, threshold metric module 330 may determine modifications to the second threshold metric using the threshold metric model. The modifications may be determined based on the use metrics for the user, the second threshold metric and/or a risk score for the user indicating risk of disengagement from usage of the wearable device. For example, if the second threshold metric is six hours of use of the wearable device, the use metrics for the user have exceeded six hours of use for 92% of the previous 60 days and the risk score indicates low risk of disengagement of the user, threshold metric module 330 may modify the second threshold metric from six hours to seven hours. But, if the use metrics for the user have exceeded six hours of use for only 20% of the previous 60 days and the risk score indicates high risk of disengagement of the user, threshold metric module 330 may modify the second threshold metric from six hours to five hours. In some examples, the modifications to the second threshold metric may need to be approved by a program provider or a program administrator before they become active and are used for subsequent comparisons.

At 560, a second indication of the second use metric and the modified second threshold metric may be provided to the user. For example, the second indication may be provided to the user on display 306 using user interface module 314 showing modifications (if any) to the usage goals.

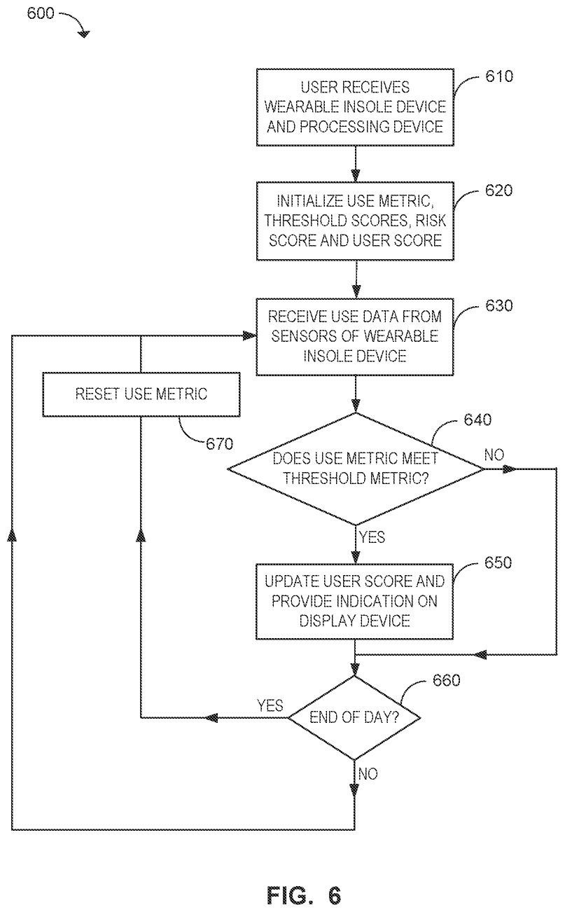

Referring now to FIG. 6, shown therein is a flowchart illustrating an example of a method 600 for analyzing the daily hours of use of a wearable insole device by a user. Method 600 can be implemented, for example, using system 100 shown in FIG. 1.

At 610, a user may receive a wearable insole device and a processing device. A healthcare practitioner, a program provider or a program administrator may provide the wearable insole device and the processing device to the user based on a health and/or fitness assessment of the user. For example, the user may receive the wearable insole device and the processing device to manage the user's DFU condition. The user may receive two wearable insole devices (one for each foot) and a single handheld processing device that can receive and analyze data from sensors of both wearable insole devices.

In some examples, the user may register with a digital app associated with the wearable insole device. The user may input their demographic and other information or provide answers to a questionnaire during the registration.

At 620, a daily hours of use metric, one or more threshold scores, a risk score, and a user score may be initialized for the user. Use metric module 326 can compute the daily hours of use metric for the user. In addition to the daily hours of use metric, use metric module 326 can also compute other use metrics indicating use of the wearable device (as described herein above). At 620, use metric module 326 may initialize the daily hours of use metric to zero.

Risk score module 332 may determine an initial risk score indicating risk of disengagement of the user from using the wearable insole device based on the information provided by the user during registration. Risk score module 332 can also determine the initial risk score based on information provided by a healthcare practitioner, a program provider or a program administrator. In some examples, the initial risk score may be provided to the processing device by a healthcare practitioner, a program provider or a program administrator. The initial risk score may be user-specific or based on a grouping of the user.

There may be multiple threshold scores associated with the daily hours of use metric. For example, a baseline threshold metric, an improved outcome threshold metric and a maximum threshold metric may be associated with the daily hours of use metric. The initial values of the threshold metrics may be provided to the processing device by a healthcare practitioner, a program provider or a program administrator. The initial threshold metrics can be user-specific or based on a grouping of the user. In some examples, threshold metric module 330 may generate the initial threshold metrics. For example, the baseline threshold metric may be 30 minutes, the improved outcome threshold metric may be 4.5 hours and the maximum threshold metric may be 8.5 hours.

There may be multiple threshold scores associated with the use metric of consecutive or non-consecutive days of use. For example, an overall threshold metric, a first check-in threshold metric, and a second check-in threshold metric may be associated with the non-consecutive days of use metric. The initial values of the threshold metrics may be provided to the processing device 108 by a healthcare practitioner, a program provider or a program administrator. The initial threshold metrics can be user-specific or based on a grouping of the user. In some examples, threshold metric module 330 may generate the initial threshold metrics. For example, the overall threshold metric may be 16 non-consecutive days of use in a month (i.e. approximately 16/30=53% non-consecutive days of use). The first check-in threshold metric and second threshold check-in metric may be pro-rated ratios to the overall threshold metric, to incentivize or alert the user to their progress in achieving the overall threshold metric by the end of the month. The first check-in metric may be 6 non-consecutive days of use by the 10^{th} day in the month (i.e. 6/10=60% non-consecutive days of use), which is the closest number of full days exceeding the pro-rated ratio for the overall threshold metric. Similarly, the second check-in metric may be 11 non-consecutive use days by the 20^{th} day in the month (i.e. 11/20=55% non-consecutive days of use).

User score module 328 may generate an initial user score associated with the user. The user score may be initialized as zero. In some examples, user score module 328 may update the user score by an initial bonus score amount in response to the user completing the registration process for the digital app associated with the wearable device.

At 630, the processing device may receive data from the sensors of the wearable insole device in response to the user using the wearable insole device. For example, the processing device may receive data from the pressure and temperature sensors of the wearable insole device indicating that the device is being used by the user. Use metric module 326 can compute the daily hours of use metric based on the received data.

At 640, the computed use metrics may be compared with the corresponding threshold metrics. For example, when the user begins use of the wearable insole device, threshold metric module 330 can compare the computed daily hours of use metric to the baseline threshold metric of 30 minutes. If the computed daily hours of use metric is less than 30 minutes (e.g., computed daily hours of use metric is 20 minutes), method 600 may continue to 660. If the computed daily hours of use metric meets the 30 minute baseline threshold metric (e.g., computed daily hours of use metric is 40 minutes), method 600 may proceed to 650.

At 650, the user score may be updated by a score amount. For example, user score module 328 may update the user score by 25 for meeting the baseline threshold metric. An indication of the updated user score may be provided to the user, for example, on display 306 using user interface module 314.

At 660, the processing device may determine if the current day has ended. If the current day has ended, use metric module 326 may reset the daily hours of use metric for the user. Different reset criteria may be used for other use metrics. For an example consecutive days of use metric corresponding to a minimum goal of 5 hours of use each day, the consecutive days of use metric may only be reset at the end of a day where the minimum goal of 5 hours is not met. Other time periods (e.g., a week, a month etc.) may also be used in the reset criteria. For example, a non-consecutive days of use metric may be reset at the end of every month where the minimum goal is 0.9 hours of use for 16 non-consecutive days. Different use metrics may have different reset criteria. In some examples, the use metrics may be determined as running averages and may not have any reset criteria associated with them. For example, an interaction level of the user with the digital app associated with the wearable device may be computed based on the interaction data of the user for the previous 60 days and may not be reset at the end of each day.

At 660, if the current day has not ended, method 600 may continue to 630 to receive further use data and update the use metric accordingly. If the baseline threshold metric has already been met, user score module 328 may update the user score by a score amount of 7.5 for each hour of additional use until the daily hours of use metric meets the improved outcome threshold metric of 4.5 hours.

If the daily hours of use metric meets the next threshold metric (in this case, the improved outcome threshold metric of 4.5 hours), user score module 328 may update the user score by a score amount of 22.5 (7.5 for the additional hour+a bonus amount of 15 for meeting the improved outcome threshold metric). An indication of the updated user score may be provided to the user, for example, on display 306.

Method 600 may continue again to 660 to check if the current day has ended. If the current day has not ended, user score module 328 may continue to update the user score by a score amount of 7.5 for each hour of additional use until the daily hours of use metric meets the maximum threshold metric of 8.5 hours. User score module 328 may update the user score by a score amount of 7.5 for meeting the maximum threshold metric of 8.5 hours (7.5 for the additional hour, but not providing any bonus score amount for meeting the maximum threshold metric). An indication of the updated user score may be provided to the user, for example, on display 306 along with a notification that the maximum threshold metric has been reached and user score will not be increased for any additional use of the wearable insole device on that day.

While the above description provides examples of one or more processes or apparatuses or compositions, it will be appreciated that other processes or apparatuses or compositions may be within the scope of the accompanying claims.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

We claim:

1. A computer-implemented method comprising:
receiving, by a processor, first use data associated with use of at least one wearable device by a user, wherein the first use data comprises data generated from one or more sensors of the at least one wearable device;
computing, by the processor, a first use metric associated with the user based on the first use data;
comparing the first use metric to a first threshold metric;
based on the comparing, updating a user score by a first score amount;
computing a second score amount and a second threshold metric; and
providing, by the processor, a first indication of the user score, the second threshold metric, and the second score amount on a display device.

2. The method of claim 1, further comprising;
receiving, by the processor, second use data generated by use of the at least one wearable device following receipt of the first indication;
computing, by the processor, a second use metric associated with the user based on the second data;
comparing the second use metric to the second threshold metric; and
when the second use metric satisfies the second threshold metric, updating the user score by the second score amount.

3. The method of claim 2, further comprising modifying, by the processor, the second threshold metric in response to computing the second use metric.

4. The method of claim 2, further comprising modifying, by the processor, the second threshold metric based on multiple use metrics associated with multiple users.

5. The method of claim 3, wherein modifying, by the processor, the second threshold metric comprises using a machine learning model.

6. The method of claim 3, further comprising providing to the user, by the processor, a second indication on the display device, wherein the second indication is based on the second use metric and the second threshold metric as modified.

7. The method of claim 1, wherein the first threshold metric and the second threshold metric are calibrated to reduce risk factors associated with development of a diabetic foot ulcer.

8. The method of claim 1, wherein the second threshold metric includes one of a health metric and a user-specific criterion.

9. The method of claim 1, further comprising computing a risk of disengagement for the user, and wherein the computing the second threshold metric is based on the risk of disengagement.

10. The method of claim 1, further comprising:
receiving, by the processor, a healthcare assessment for the user for a time period associated with use of the at least one wearable device; and
modifying, by the processor, the second threshold metric in response to the input and based on at least one of the first use metric and the second use metric.

11. The method of claim 1, wherein the first use data indicates one or more of a duration of wear of the at least one wearable device by the user, a number of consecutive or non-consecutive days of use of the at least one wearable device by the user, a duration of wear of the at least one wearable device by the user within a predetermined time interval, a response of the user to one or more alerts provided by the at least one wearable device, a response of the user to one or more indications provided by the processor, an activity level of the user, an interaction level of the user with a digital platform associated with the at least one wearable device, a use level of a wireless charger for the at least one wearable device by the user, and a care level of the at least one wearable device by the user.

12. A wearable monitoring system comprising:
at least one wearable device comprising one or more sensors; and
a computing device comprising a processor, a memory, and a display device, wherein the processor is configured to:
receive first use data, the first use data associated with use of at least one wearable device by a user, wherein the first use data comprises data generated from the one or more sensors of the at least one wearable device;
compute a first use metric associated with the user based on the first use data;
compare the first use metric to a first threshold metric;
based on the comparing, update a user score by a first score amount;
compute a second score amount and a second threshold metric; and
provide a first indication of the user score, the second threshold metric, and the second score amount on the display device.

13. The wearable monitoring system of claim 12, wherein the processor is further configured to:
receive second use data generated by use of the at least one wearable device following receipt of the first indication;
compute a second use metric associated with the user based on the second data;
compare the second use metric to the second threshold metric; and
when the second use metric satisfies the second threshold metric, update the user score by the second score amount.

14. The wearable monitoring system of claim 13, wherein the processor is further configured to modify the second threshold metric in response to computing the second use metric.

15. The wearable monitoring system of claim 12, wherein the at least one wearable device is a sensorized insole.

16. The wearable monitoring system of claim 12, wherein the first threshold metric and the second threshold metric are calibrated to reduce risk factors associated with development of a diabetic foot ulcer.

17. The wearable monitoring system of claim 12, wherein the processor is further configured to compute a risk of disengagement for the user, and wherein the computing the second threshold metric is based on the risk of disengagement.

18. The wearable monitoring system of claim 13, wherein the first indication includes at least one of the first use metric, and the second use metric.

19. The wearable monitoring system of claim 12, wherein the one or more sensors of the at least one wearable device includes at least one of a force sensor and a temperature sensor.

20. A wearable monitoring system comprising:
at least one wearable device comprising one or more sensors; and
a computing device comprising a processor, a memory, and a display device, wherein the processor is configured to:
receive sensor data and use data, the sensor data and the use data associated with use of the at least one wearable device by a user, wherein the sensor data and the use data comprise data generated from the one or more sensors of the at least one wearable device;
generate monitoring data, the monitoring data generated from monitoring the sensor data and the use data of the at least one wearable device of the user;
compare a numeric value of a day of a specified period to a numeric value of a predefined day of the specified period;
compute a use metric based on the sensor data or the use data or a monitoring metric based on the monitoring data, if the numeric value of the day of the specified period is greater than or equal to the numeric value of the predefined day of the specified period;
compare the use metric or the monitoring metric to a threshold metric;
determine whether the use metric or the monitoring metric satisfies a threshold requirement of the threshold metric; and
generate an alert on the display device if the use metric or the monitoring metric does not satisfy the threshold requirement.
